(19) United States
(12) Patent Application Publication
(10) Pub. No.: US 2007/0299091 A1
(43) Pub. Date: Dec. 27, 2007

(54) Azaindole Carboxamides

(76) Inventors: Peter Gmeiner, Buckenhof (DK); Karin Schlotter, Oettingen (DE); Harald Huber, Heroldsbach (DE); Dirk Schmidt, Monheim (DE); Monika Buchholz, Langenfeld (DE)

Correspondence Address: HARNESS, DICKEY & PIERCE, P.L.C., P.O. BOX 828, BLOOMFIELD HILLS, MI, 48303, US

(21) Appl. No.: 11/667,601

(22) PCT Filed: Nov. 11, 2005
(86) PCT No.: PCT/EP05/12127

§ 371 Date: May 11, 2007

(30) Foreign Application Priority Data
Nov. 12, 2004 (DE) 10 2004 054 634.7

(51) Int. Cl. C07D 471/04; A61K 31/437; C07D 487/04; A61K 31/519

(52) U.S. Cl. 514/265.1; 514/300; 514/303; 544/280; 546/113; 546/118

(57) ABSTRACT

The invention relates to azaindole derivatives of general formula (I), 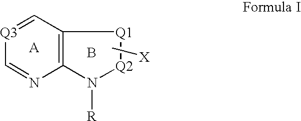
wherein X represents a group of general formula (X1). 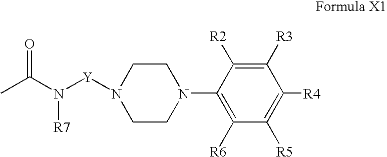 Said compounds have a therapeutic potential in the treatment of diseases that are accompanied by an impaired dopamine metabolism and/or abnormal serotonin-5-HT1a signal transmission.

Dopamine is an important neurotransmitter of the central nervous system. Dopamine is effective by binding to five different dopamine receptors. As a result of their morphology and the nature of their signal transmission these can be classified as D1-like (D1 and D5) and D2-like (D2-, D3- and D4-receptors) (Neve, K. A. The Dopamine Receptors. Humana Press, 1997). The sub-types of the D2 family in particular have an important part to play in the regulation of central nervous processes. While the D2-receptors are predominantly expressed in the basal ganglions and are involved there in the control and modulation of neuromotor circuits, D3-receptors are mainly found in the mesolimbic system, in which emotional and cognitive processes are controlled. Disturbances in the signal transduction of these receptors lead to a number of neuropathological changes which can sometimes result in serious illnesses. As a result the D3-receptor in particular is a promising target for the development of active substances for the treatment of psychiatric illnesses such as schizophrenia or unipolar depressions, of disturbances of consciousness and for treatment of neurodegenerative diseases such as Parkinson's and the dyskinesia that can occur in the course of long-term therapy, but also for the treatment of drug dependency (Pulvirenti, L. et al. Trends Pharmacol. Sci. 2002, 23,151-153, Joyce, J. N. Pharmacol. Ther. 2001, 90, 231-259). Here the most D3-receptor-selective bonding profile should be sought. Depending on the intrinsic activity (full agonist, partial agonist, antagonist or inverse agonist) such ligands can have a stimulating, modulating or also inhibiting effect on the pathologically altered dopamine signal transduction system and can thus be used for the treatment of these diseases.

Compounds with an arylpiperazine structure have previously been described as dopamine receptor-active ligands (Robarge, M. J. J. Med. Chem. 2001, 44, 3175-3186). Benzamides and naphthamides with arylpiperazine partial structures are also known as ligands of dopamine receptors (Perrone, R. J. Med. Chem. 1998, 41, 4903-4909; EP 0 779 284 A1). Recently heteroarene amides have also been described as D3-receptor-active compounds (Bettinetti, L. et al. J. Med. Chem. 2002, 45, 4594-4597, Leopoldo, M. et al. J. Med. Chem. 2002, 45, 5727-5735, WO 2004004729 A1). A phenylpiperazinylnaphthamide has also recently been reported on as a selective D3-partial agonist, which demonstrated hopeful activities in the animal model, and which could be used for the treatment of cocaine addiction (Pilla, M. et al. Nature 1999, 400, 371-375). Furthermore, because of the characteristic features of this compound elimination of the serious motor impairments (dyskinesias) caused by long-term treatment of Parkinson's disease with the pharmaceutical preparation L-DOPA can be achieved (Bezard, E. et al. Nature Med. 2003, 9, 762-767). The most recent literature describes the neuro-protective effect of D3-selective partial agonists against MPTP-induced neurone loss in mice as a murine model for Parkinson's disease (Boeckler, F. et al. Biochem. Pharmacol. 2003, 6, 1025-1032).

Of the range of arylpiperazinylheteroarene carboxamides structural examples with oxygen-, sulphur- or nitrogen-containing heteroarene carboxylic acid components are above all described (ES 2027898; EP 343 961; U.S. Pat. No. 3,646,047; U.S. Pat. No. 3,734,915; WO 2004/024878; Leopoldo, M. et al. J. Med. Chem. 2002, 45, 5727-5735, Bettinetti, L. et al. J. Med. Chem. 2002, 45, 4594-4597; WO 2004004729 A1).

The structural characteristic shared by many high affinity dopamine receptor ligands concerns a variable substituted phenylpiperazine partial structure, which is linked via a spacer of several carbons in length to an aryl- or heteroarylcarboxamide. Such compounds are, by way of example, described in Bettinetti, L. et al. J. Med. Chem. 2002, 45, 4594-4597, Campiani, G. et al. J. Med. Chem. 2003, 46, 3822-3839 and Hackling, A. et al. J. Med. Chem. 2003, 46, 3883-3889.

To date only carboxamide-substituted heteroaromatic systems have been described which have a heteroatom in the pentacycle. Heteroatoms in the aromatic hexacycle have to date only been known in compounds from the prior art which have a nitrogen atom in the annulation position of the bicycle, like for example pyrazolo[1,5-a]pyridines. However a nitrogen atom in said annulation position has no basic characteristics.

In the context of intensive structure-effect investigations into dopamine receptor ligands, it has now surprisingly been found that the dopamine D3-receptor also recognises hereoarene carboxamides as high affinity ligands which contain a nitrogen atom with basic characteristics in the six-membered aromatic ring system.

The subject-matter of the invention thus comprises azaindoles with a basic nitrogen in the six-ring of the heterocycle, which in the 2 or 3 position of the 5-ring are substituted with a carboxamide unit. During in vitro research these demonstrated a high affinity and selective binding characteristics to the D3-receptor. Some compounds also demonstrate a notable affinity to serotoninergic receptors, in particular to the 5-HT1 a-receptor.

The compounds according to the invention could therefore constitute valuable therapeutic agents for the treatment of central nervous system disorders, such as for example schizophrenia or various types of depression, for neuroprotection in neurodegenerative diseases, in addictive disorders, glaucoma, cognitive disorders, restless leg syndrome, attention deficit hyperactive syndrome (ADHS), hyperprolactinemia, hyperprolactinomia and autism, in idiopathic or medically-induced extrapyramidal motor disturbances, such as akathisia, rigor, dystonia and dyskinesias, as well as various disorders of the urinary tract.

Subject matter of this invention is compounds of the general formula I,

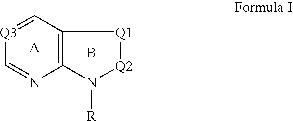

in which:

A is an aromatic 6-membered ring, the ring-forming C-atoms of which in each case and independently of one another can carry a substituent R1;

B is an aromatic 5-membered ring, which carries exactly one X group;

Q1 is N, N—R′; S, O, CH, C—R1 or C—X;

Q2 is CH, C—R1 or C—X, wherein either Q1 or Q2 form a C—X group;

Q3 is N, CH or C—R1;

R1 is in each case independently selected from among hydroxy, alkyl, alkyloxy, alkylthio, alkenyl, alkinyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino;

R′ is selected from among hydrogen, alkyl, phenyl, phenylalkyl, alkylcarbonyl, phenylcarbonyl, phenylalkylcarbonyl and phenylsulfonyl;

R is absent if Q1 represents N—R′, S or O; or R is selected from among hydrogen, alkyl, phenyl, phenylalkyl, alkylcarbonyl, phenylcarbonyl, phenylalkylcarbonyl and phenylsulfonyl, if Q1 is N, CH, C—R1 or C—X.

X is a group of general formula X1

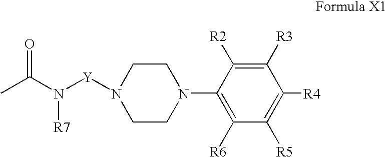

wherein:

Y is an unbranched, saturated or unsaturated hydrocarbon chain with 2-5 carbon atoms or a chain —(CH₂)ₒ-Z-(CH₂)ₚ—, in which Z is selected from the residues cyclopentyl, cyclohexyl and cycloheptyl, wherein o and p in each case and independently of one another have the value 0, 1, 2 or 3 and wherein the sum of o and p is at most 3;

R2, R3, R4, R5 and R6 are in each case and independently of one another selected from hydrogen, hydroxy, alkyl, alkyloxy, alkylthio, alkenyl, alkynyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, and wherein two vicinal residues R2, R3, R4, R5 and R6 together with the C-atoms of the phenyl ring to which they are bonded can form an oxygen-containing 5-, 6- or 7-membered ring;

R7 is alkyl or preferably hydrogen;

in the form of the free base, their physiologically acceptable salts and possible enantiomers, diastereomers and tautomers.

In one embodiment of the invention the two rings A and B, apart from the X group, have a maximum of 3, 2 or 1 substituents R1 or are unsubstituted apart from the X group.

In a preferred embodiment of the invention the substituents R1 of the heteroarene in the compounds according to the invention of general formula I are selected from hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methyoxy groups; phenyl(C1-C6)alkyl, wherein the phenyl is unsubstituted or substituted with fluorine, chlorine or bromine and/or with one or more methoxy groups, and wherein the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkylaminosulfonyl, in particular methylaminosulfonyl and C1-C6 alkylsulfonylamino; in particular methanesulfonylamino.

In the compounds according to the invention of general formula I, R2, R3, R4, R5 and R6 are preferably and independently of one another selected from the group comprising hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methyoxy groups; phenyl(C1-C6)alkyl, wherein the phenyl is unsubstituted or substituted with fluorine, chlorine or bromine and/or with one or more methoxy groups substituted and wherein the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups and wherein the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkylaminosulfonyl, in particular methylaminosulfonyl and C1-C6 alkylsulfonylamino; in particular methanesulfonylamino, or where two vicinal residues R2, R3, R4, R5 and R6 together with the C-atoms of the phenyl ring to which they are bonded form an oxygen-containing 5-, 6- or 7-membered ring.

In a preferred embodiment of the invention Y in the compounds according to the invention is a chain —(CH₂)ₚ-Z-(CH₂)ₒ—, wherein Z is selected from the residues cyclopentyl, cyclohexyl and cycloheptyl, and wherein p and o are independently of one another selected from 0, 1 and 2 and together provide a maximum value of 2 or 1 or are both 0.

In the compounds of general formula I or X1, Y is preferably a hydrocarbon chain of formula —(CH₂)_{q}— with q=2, 3, 4 or 5, particularly preferably with n=4 or 5.

X thus particularly preferably represents a group of general formula X2

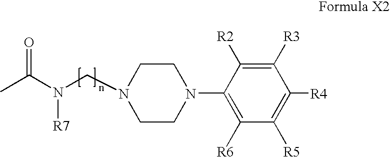

in which n has the value 2-5 and particularly preferably the value 4 or 5, and the substituents R2, R3, R4, R5, R6 and R7 have the meaning described in more detail above.

In a preferred embodiment at least one of the two residues R2 and R3 stands for a substituent other than hydrogen, in particular for halogen or C1-C6 alkyl or C1-C6 alkyloxy, while the residues R4, R5 and R6 in the compounds according to the invention of general formula I or in formula X1 and formula X2 in each case stand for hydrogen.

In a preferred embodiment of the invention one of the two substituents R2 or R3 is a halogen, in particular fluorine or chlorine, particularly preferably R2 and R3 both being halogen, most particularly preferably chlorine.

In a further preferred embodiment of the invention two vicinal substituents selected from R2, R3, R4, R5 and R6, and in particular substituents R2 and R3 together with the phenyl residue to which they are bonded form a chromane, tetrahydrobenzoxepine or dihydrobenzofuran in the compounds of general formula I.

A further preferred aspect of the present invention concerns compounds of general formula I in embodiments as described in the following under “Formula 1a”:

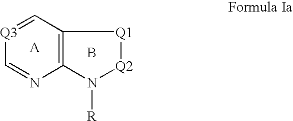

wherein:

A is an aromatic 6-membered ring, the ring-forming C-atoms of which in each case and independently of one another can carry a substituent R1;

B is an aromatic 5-membered ring, which carries precisely one X group;

Q1 is N, N—R′; CH, C—R1 or C—X;

Q2 is CH, C—R1 or C—X, wherein either Q1 or Q2 form a C—X group;

Q3 is N, CH or C—R1;

R1 is in each case in the compounds of general formula Ia independently selected from the group hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups, and wherein the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or substituted with one or more methoxy groups, and wherein the C1-C6 alkyl is unsubstituted or hydroxy substituted; C1-C6 alkylaminosulfonyl, in particular methylaminosulfonyl and C1-C6 alkylsulfonylamino; in particular methanesulfonylamino;

R′ is selected from among hydrogen; unsubstituted or hydroxy substituted C1-C6 alkyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups, and wherein the C1-C6 alkyl is unsubstituted or hydroxy substituted; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or substituted with one or more methoxy groups and wherein the C1-C6 alkyl is unsubstituted or hydroxy substituted; and phenylsulfonyl, wherein the phenyl is unsubstituted or substituted with fluorine, chlorine or bromine and/or substituted with one or more methoxy groups;

if Q1 represents N—R′, R is absent;

if Q1 represents N, CH, C—R1 or C—X, R is selected from the group of hydrogen; unsubstituted or hydroxy substituted C1-C6 alkyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups, and wherein the C1-C6 alkyl is unsubstituted or hydroxy substituted; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or substituted with one or more methoxy groups and wherein the C1-C6 alkyl is unsubstituted or hydroxy substituted; and phenylsulfonyl, wherein the phenyl is unsubstituted or substituted with fluorine, chlorine or bromine and/or substituted with one or more methoxy groups;

X is in compounds of general formula 1a a group of general formula X2

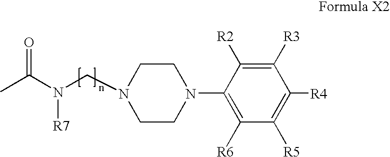

in which n has the value 2-5 particularly preferably the value 4 or 5 and in which the substituents R2, R3, R4, R5 R6 and R7 preferably and in each case independently of one another are selected from the group of hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methyoxy groups; phenyl(C1-C6)alkyl, wherein the phenyl is unsubstituted or substituted with fluorine, chlorine or bromine and/or with one or more methoxy groups, and wherein the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or with fluorine, chlorine or bromine and/or with one or more methoxy groups substituted and wherein the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkylaminosulfonyl, in particular methylaminosulfonyl and C1-C6 alkylsulfonylamino; in particular methanesulfonylamino, or two vicinal residues R2, R3, R4, R5 and R6 together with the C-atoms of the phenyl ring to which they are bonded form an oxygen-containing 5-, 6- or 7-membered ring.

R7 is C1-C6 alkyl or, preferably, hydrogen;

in the form of the free base, their physiologically acceptable salts and possible enantiomers, diastereomers and tautomers.

Example compounds of formulae I or Ia are selected from among

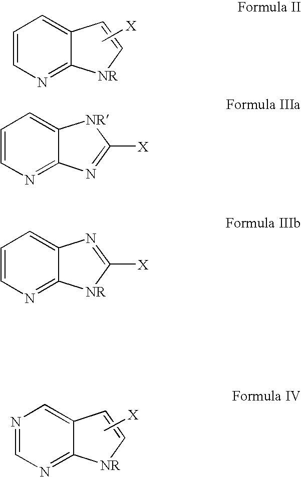

wherein

R, R′ and X in each case have the significance described in more detail above under formulae I and Ia and the C-atoms of the ring A can in each case and independently of one another carry a substituent R1, as defined above under formulae I and Ia.

In a preferred embodiment the invention concerns compounds of general formula II

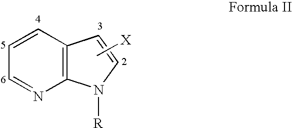

in which:

the substituent X is linked with position 2 or 3 of the pyrrolo[2,3-b]pyridine and represents a group as described in more detail above under formula I or formula Ia;

the pyrrolo[2,3-b]pyridine can in positions 4-6 of the A ring or at the position 2 or 3 of the B ring not linked with X in each case carry substituents R1, as described in more detail above under formula I or formula Ia, wherein the pyrrolo[2,3-b]pyridine preferably has a maximum of two substituents R1 and particularly preferably is unsubstituted;

R is a group as described above under formula I or formula Ia and is preferably a hydrogen atom, a methyl group or a phenylsulfonyl;

the substituent X in the compounds of general formula II is preferably in the form of a group of general formula X2

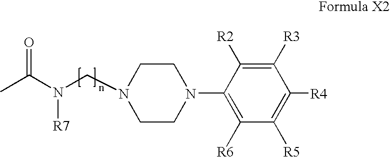

in which:

n is 2, 3, 4 or 5, particularly preferably 4 or 5;

R2, R3, R4, R5, R6 and R7 are substituents, as described above under formula I or formula Ia; in preferred embodiments R4, R5 and R6 are in each case hydrogen, while R2 and R3 are by way of example selected from among hydrogen, chlorine, fluorine, methoxy, ethoxy, propoxy, methyl, ethyl and propyl; in another preferred embodiment the invention concerns compounds of general formula II, wherein at least one of the substituents R2 or R3 is selected from among chlorine, fluorine, methoxy, ethoxy, propoxy, methyl, ethyl and propyl.

Examples of compounds are

N-(4-(4-(2-methoxyphenyl)piperazin-1-yl)butyl)-1H-pyrrolo[2,3-b]pyridin-2-ylcarbamide,

N-(4-(4-(2-methoxyphenyl)piperazin-1-yl)butyl)-1-phenylsulfonyl-1H-pyrrolo[2,3-b]pyridin-2-ylcarbamide,

N-(4-(4-(2,3-dichlorophenyl)piperazin-1-yl)butyl)-1H-pyrrolo[2,3-b]pyridin-2-ylcarbamide,

N-(4-(4-(2,3-dichlorophenyl)piperazin-1-yl)butyl)-1H-1-phenylsulfonylpyrrolo[2,3-b]pyridin-2-ylcarbamide,

N-(4-(4-(2,3-dichlorophenyl)piperazin-1-yl)butyl)-1H-pyrrolo[2,3-b]pyridin-3-ylcarbamide,

N-(4-(4-(2-methoxyphenyl)piperazin-1-yl)butyl)-1-methyl-1H-pyrrolo[2,3-b]pyridin-3-ylcarbamide,

N-(4-(4-(2,3-dichlorophenyl)piperazin-1-yl)butyl)-1-phenylsulfonyl-1H-pyrrolo[2,3-b]pyridin-3-ylcarbamide,

N-(4-(4-(2-methoxyphenyl)piperazin-1-yl)butyl)-1-phenylsulfonyl-1H-pyrrolo[2,3-b]pyridin-3-ylcarbamide,

N-(4-(4-(2-ethoxyphenyl)piperazin-1-yl)butyl)-1-phenylsulfonyl-1H-pyrrolo[2,3-b]pyridin-3-ylcarbamide,

N-(4-(4-(2,3-dimethylphenyl)piperazin-1-yl)butyl)-1-phenylsulfonyl-1H-pyrrolo[2,3-b]pyridin-3-ylcarbamide,

N-(4-(4-(dihydrobenzofuran-7-yl)piperazin-1-yl)butyl)-1H-pyrrolo[2,3-b]pyridin-2-ylcarbamide,

N-(4-(4-(dihydrobenzofuran-7-yl)piperazin-1-yl)butyl)-1-phenylsulfonyl-1H-pyrrolo[2,3-b]pyridin-2-ylcarbamide,

N-(4-(4-(chroman-8-yl)piperazin-1-yl)butyl)-1H-pyrrolo[2,3-b]pyridin-2-ylcarbamide,

N-(4-(4-(chroman-8-yl)piperazin-1-yl)butyl)-1-phenylsulfonyl-1H-pyrrolo[2,3-b]pyridin-2-ylcarbamide,

N-(4-(4-(2,3,4,5-tetrahydrobenzo[b]oxepin-9-yl)piperazin-1-yl)butyl)-1H-pyrrolo[2,3-b]pyridin-2-ylcarbamide,

N-(4-(4-(2,3,4,5-tetrahydrobenzo[b]oxepin-9-yl)piperazin-1-yl)butyl)-1-phenylsulfonyl-1H-pyrrolo[2,3-b]pyridin-2-ylcarbamide,

N-(4-(4-(dihydrobenzofuran-7-yl)piperazin-1-yl)butyl)-1H-pyrrolo[2,3-b]pyridin-3-ylcarbamide,

N-(4-(4-(dihydrobenzofuran-7-yl)piperazin-1-yl)butyl)-1-phenylsulfonyl-1H-pyrrolo[2,3-b]pyridin-3-ylcarbamide,

N-(4-(4-(chroman-8-yl)piperazin-1-yl)butyl)-1H-pyrrolo[2,3-b]pyridin-3-ylcarbamide,

N-(4-(4-(chroman-8-yl)piperazin-1-yl)butyl)-1-phenylsulfonyl-1H-pyrrolo[2,3-b]pyridin-3-ylcarbamide,

N-(4-(4-(2,3,4,5-tetrahydrobenzo[b]oxepin-9-yl)piperazin-1-yl)butyl)-1H-pyrrolo[2,3-b]pyridin-3-ylcarbamide, and

N-(4-(4-(2,3,4,5-tetrahydrobenzo[b]oxepin-9-yl)piperazin-1-yl)butyl)-1-phenylsulfonyl-1H-pyrrolo[2,3-b]pyridin-3-ylcarbamide.

In another preferred embodiment the invention concerns compounds of general formula IIIa or IIIb

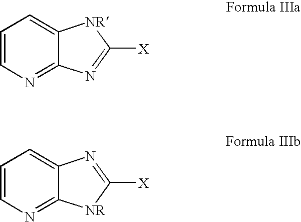

in which:

the substituent X represents a group, as defined in more detail above under formula I or formula Ia;

the imidazo[4,5-b]pyridine can in the A ring carry one or more substituents R1, as described in more detail above under formula I or formula Ia, wherein the A ring preferably carries a maximum of two substituents R1 and in a preferred embodiment is unsubstituted;

R and R′ are groups, as described in more detail above under formula I or formula Ia.

A preferred embodiment of the invention concerns compounds of formula IIIb, in particular if the substituent R is a hydrogen atom or a phenylsulfonyl.

In a further preferred embodiment of the invention the substituent X in the compounds of general formula III, in particular the compounds of formula IIIb, is in the form of a group of general formula X2

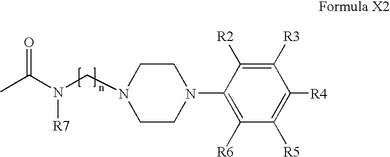

in which:

n is 2, 3, 4 or 5 and particularly preferably 4 or 5;

R2, R3, R4, R5, R6 and R7 are substituents as described above under formula I or formula Ia; in preferred embodiments R4, R5 and R6 are in each case hydrogen, while R2 and R3 are by way of example selected from among chlorine, fluorine, methoxy, ethoxy, propoxy, methyl, ethyl and propyl; in another preferred embodiment the invention concerns compounds of general formula III, wherein at least one of the substituents R2 or R3 is a methoxy group or a halogen atom. In another embodiment the substituent R4 is a substituent other than hydrogen, e.g. fluorine.

Examples of compounds of formula III are

N-(4-(4-(2-methoxyphenyl)piperazin-1-yl)butyl)-3H-imidazo[4,5-b]pyridin-2-ylcarbamide,

N-(4-(4-(2,3-dichlorophenyl)piperazin-1-yl)butyl)-3H-imidazo[4,5-b]pyridin-2-ylcarbamide,

N-(4-(4-(2-chlorophenyl)piperazin-1-yl)butyl)-3H-imidazo[4,5-b]pyridin-2-ylcarbamide,

N-(4-(4-(2,3-dimethylphenyl)piperazin-1-yl)butyl)-3H-imidazo[4,5-b]pyridin-2-ylcarbamide,

N-(4-(4-(4-fluorophenyl)piperazin-1-yl)butyl)-3H-imidazo[4,5-b]pyridin-2-ylcarbamide,

N-(4-(4-(2,3-dihydrobenzofuran-7-yl)piperazin-1-yl)butyl)-3H-imidazo[4,5-b]pyridin-2-ylcarbamide,

N-(4-(4-(chroman-8-yl)piperazin-1-yl)butyl)-3H-imidazo[4,5-b]pyridin-2-ylcarbamide, and

N-(4-(4-(2,3,4,5-tetrahydrobenzo[b]oxepin-9-yl)piperazin-1-yl)butyl)-3H-imidazo[4,5-b]pyridin-2-ylcarbamide.

In another preferred embodiment the invention concerns compounds of general formula IV

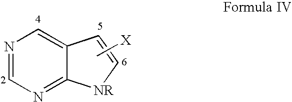

in which:

the substituent X is linked in positions 5 or 6 with the heteroarene core and represents a group as described in more detail above under formula I or formula Ia;

the pyrrolo[2,3-b]pyrimidine can in positions 2 and 4 of the A ring or at the position 5 or 6 of the B ring not linked with X in each case carry substituents R1, as described in more detail above under formula I or formula Ia; in examples of embodiments a compound of formula IV carries one or two substituents R1 selected from among hydroxy and C1-C3 alkyl; in another embodiment the pyrrolo[2,3-b]pyrimidine carries no substituents R1;

R is in compounds of general formula IV a group, as described in more detail above under formula I or formula Ia and preferably represents hydrogen, phenyl sulfonyl or a phenyl which is unsubstituted or substituted with one or more halogen atoms.

In a further preferred embodiment of the invention the substituent X in the compounds of general formula IV, in particular the compounds of formula IIIb, is in the form of a group of general formula X2

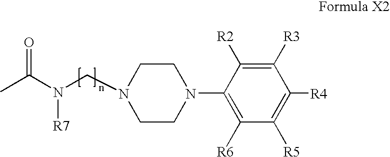

in which:

n is 2, 3, 4 or 5 and particularly preferably 4 or 5;

R2, R3, R4, R5, R6 and R7 are substituents, as described above under formula I or formula Ia; in preferred embodiments R4, R5 and R6 are in each case hydrogen, while at least one of the substituents R2 and R3 is by way of example selected from among chlorine, fluorine, methoxy, ethoxy, propoxy, methyl, ethyl and propyl; in a preferred embodiment the invention concerns compounds of general formula IV, wherein at least one of the substituents R2 or R3 is a methoxy group or a halogen atom.

Examples of compounds of formula IV are

N-(4-(4-(2,3-dichlorophenyl)piperazin-1-yl)butyl)-5-methyl-4-hydroxy-7-phenyl-7H-pyrrolo[2,3-d]pyrimidin-6-ylcarbamide and tautomers of this

N-(4-(4-(2-methoxyphenyl)piperazin-1-yl)butyl)-5-methyl-4-hydroxy-7-phenyl-7H-pyrrolo[2,3-d]pyrimidin-6-ylcarbamide and tautomers of this

N-(4-(4-(2,3-dihydrobenzofuran-7-yl)piperazin-1-yl)butyl)-5-methyl-4-hydroxy-7-phenyl-7H-pyrrolo[2,3-d]pyrimidin-6-ylcarbamide and tautomers of this

N-(4-(4-(chroman-8-yl)piperazin-1-yl)butyl)-5-methyl-4-hydroxy-7-phenyl-7H-pyrrolo[2,3-d]pyrimidin-6-ylcarbamide and tautomers of this

N-(4-(4-(2,3,4,5-tetrahydrobenzo[b]oxepin-9-yl)piperazin-1-yl)butyl)-5-methyl-4-hydroxy-7-phenyl-7H-pyrrolo[2,3-d]pyrimidin-6-ylcarbamide and tautomers of this.

The invention also concerns physiologically acceptable salts of the compounds according to the invention. Examples of such salts are described in the following definitions.

To the person skilled in the art it is clear that depending on the choice of substituents geometrical isomers and/or optically active compounds can result. In this case both the isomers and racemates and also the respective pure enantiomeric or possibly diastereomeric forms are the subject matter of the present invention. The invention also covers tautomers of the disclosed compounds. For example, it will be clear to the person skilled in the art that a hydroxy group can be present in a (hetero)aromatic ring through tautomerism as an oxogroup.

The substituents mentioned in the description and in the attached claims include in particular the following groups.

“Alkyl” can be a branched or unbranched alkyl group, which preferably has between 1 and 10 C-atoms, particularly preferably between 1 and 6 C-atoms (“C1-C6 alkyl”) and most particularly preferably 1, 2 or 3 C-atoms. “C1-C6 alkyl” includes, for example, methyl, ethyl, n-propyl, iso-propyl, n-butyl, iso-butyl, s-butyl, t-butyl, n-pentyl, iso-pentyl, neopentyl, t-pentyl, 1-methylbutyl, 2-methylbutyl, 1-ethylpropyl, 1,2-dimethylpropyl and n-hexyl. “Alkyl” can also be cyclical or contain a cyclical component, wherein cycles with 3-7 C-atoms are preferred, e.g. cyclopropyl, cyclobutyl, cyclopentyl, cyclohexyl or cycloheptyl. “Alkyl” is preferably not cyclical and contains no cyclical component. Alkyl groups can also be substituted with one or more substituents, in particular with hydroxy or amine. “Alkyl” is preferably unsubstituted or hydroxy substituted.

“Alkenyl” and “alkynyl” have at least one double or triple bond. They can be branched or linear and preferably have between 2 and 6 C-atoms. Alkenyls or alkynyls are preferably bonded to the heteroarene- or phenyl ring of the matrix of the compound in such a way that the double or triple bond is conjugated with the aromatic ring. Alkenyl and alkynyl can also be substituted with one or more substituents, preferably with phenyl, wherein the phenyl group then is preferably located at C-Atom 2 (if the alkenyl or alkynyl is bonded via C-atom 1 to the heteroarene- or phenyl ring of the scaffold). The alkenyls or alkynyls are preferably unsubstituted.

“Alkyloxy” is the —O-alkyl group, in which the alkyl is preferably selected from the groups specified above for “alkyl”. “Alkyloxy” is preferably a C1-C6-alkyloxy group, particularly preferably methoxy.

“Alkylthio” is the —S-alkyl group, in which the alkyl is preferably selected from the groups specified above for “alkyl”. “Alkylthio” is preferably a C1-C6-alkyl-S-group.

“Alkylaminosulfonyl” includes the —SO₂—NH-alkyl and —SO₂—N-dialkyl groups, in which alkyl is preferably selected from the groups specified above for “alkyl”. “Alkyl” in the “alkylaminosulfonyl” is preferably a C1-C6-alkyl group. “Alkylaminosulfonyl” examples include methylaminosulfonyl, N,N-dimethylaminosulfonyl or butylaminosulfonyl.

“Alkylsulfonylamino” is the —NH—SO₂-alkyl group, in which alkyl is preferably selected from the groups specified above for “alkyl”. “Alkylsulfonylamino” is preferably a C1-C6-alkylsulfonylamino group, e.g. methanesulfonylamino.

“Phenyl” is preferably unsubstituted, but can also be independently substituted one or more times, e.g. with alkoxy, alkyl, trifluoromethyl or halogen.

“Phenylalkyl” is the -alkyl-phenyl group, wherein phenyl and alkyl have the significance as defined above. Phenylalkyl includes for example phenylethyl and benzyl and is preferably benzyl.

“Phenoxy” is the —O-phenyl group, in which phenyl has the significance defined in more detail above.

“Alkylcarbonyl” includes the —C(O)-alkyl group, in which alkyl is preferably selected from the groups specified above for “alkyl”, and is particularly preferably —C(O)—C1-C6-alkyl. “Alkylcarbonyl” is preferably acetyl, propionyl or butyryl.

“Phenylcarbonyl” is —C(O)-phenyl, in which phenyl has the significance as defined in more detail above.

“Phenylalkylcarbonyl” is —C(O)-alkyl-phenyl, in which alkyl and phenyl have the significance as defined in more detail above.

“Alkyloxycarbonyl” is the —C(O)—O-alkyl group, in which alkyl is preferably selected from the groups specified above for “alkyl”. “Alkoxycarbonyl” is preferably a (C1-C6-alkyl)oxycarbonyl group.

“Phenylalkyloxycarbonyl’ is the —C(O)—O-alkyl-phenyl group, in which alkyl and phenyl have the significance as defined in more detail above.

“Halogen” includes fluorine, chlorine, bromine and iodine, and is preferably fluorine, chlorine or bromine.

“Sulfamoyl” includes the —SO₂—NH₂ group.

“Sulfonylamino” includes the —NH—SO₂H group.

“Physiologically acceptable salts” include non-toxic addition salts of a base, in particular a compound of formulae (I) to (IV) in the form of the free base, with organic or inorganic acids. Examples of inorganic acids include HCl, HBr, sulphuric acid and phosphoric acid. Organic acids include acetic acid, propionic acid, pyruvic acid, butyric acid, α-, β- or γ-hydroxbutyric acid, valeric acid, hydroxyvaleric acid, caproic acid, hydroxycaproic acid, caprylic acid, capric acid, lauric acid, myristic acid, palmitic acid, stearic acid, glycolic acid, lactic acid, D-glucuronic acid, L-glucoronic acid, D-galacturonic acid, glycine, benzoic acid, hydroxybenzoic acid, gallic acid, salicylic acid, vanillic acid, coumarinic acid, caffeic acid, hippuric acid, orotic acid, L-tartaric acid, D-tartaric acid, D,L-tartaric acid, meso-tartaric acid, fumaric acid, L-malic acid, D-malic acid, D,L-malic acid, oxalic acid, malonic acid, succinic acid, maleic acid, oxalo-acetic acid, glutaric acid, hydroxyglutaric acid, ketoglutaric acid, adipinic acid, ketoadipinic acid, pimelic acid, glutamic acid, aspartic acid, phthalic acid, propanetricarboxylic acid, citric acid, isocitric acid, methane sulfonic acid, toluene sulfonic acid, benzene sulfonic acid, camphor sulfonic acid, embonic acid and trifluoromethane sulfonic acid.

Compounds of formulae (I) to (IV) as defined, are suitable as pharmaceutical preparations. The compounds according to the invention comprise affine or even highly affine ligands for D3 receptors.

The term “medium affinity D3-ligand” covers compounds which in a radioligand experiment demonstrate bonding (see Hübner, H. et al. J. Med. Chem. 2000, 43, 756-762 and the section on “Biological Activity”) to human dopamine D3-receptors with a Ki-value of not more than 500 nM. For “medium affinity” ligands of other receptors the definition applies by analogy.

The term “high affinity D3-ligands” covers compounds which in a radioligand experiment demonstrate bonding (see Hübner, H. et al. J. Med. Chem. 2000, 43, 756-762 and the section on “Biological Activity”) to human dopamine D3-receptors with a Ki-value of preferably not more than approximately 30 nM, particularly preferably not more than 3 nM. For “high affinity” ligands of other receptors the definition applies by analogy.

One aspect of the present invention concerns selective D3-ligands. The term “selective D3-ligands” covers compounds that in the radioligand experiment for the D3-receptor, as described in the following section “Biological Activity”, have a Ki value that is lower by a factor of at least 10 than for at least five of the following seven receptors: dopamine receptors D1, D2long, D2short and D4.4, serotonin receptors 5-HT1A and 5-HT2 and alpha 1 adrenoceptor.

Another aspect of the invention concerns highly selective dopamine D3-ligands. The term “highly selective D3-ligands” covers compounds which in the radioligand experiment for the D3-receptor, as described in the following section “Biological Activity”, have a Ki-value which is lower by a factor of at least 100 than for at least three, preferably all, of the dopamine receptors D1, D2long, D2short and D4.4.

D3-ligands can have an agonistic, antagonistic or partial agonistic effect on the D3-receptor. The corresponding intrinsic activities of the compounds according to the invention can be measured in mitogenesis assays, as described in the literature (Hübner, H. et al. J. Med. Chem. 2000, 43, 4563-4569 and Löber S., Bioorg. Med. Chem. Lett. 2002, 12.17, 2377-2380). Depending on the pathophysiology of the underlying illness a stronger agonistic, a stronger antagonistic or a partial agonistic activity may be therapeutically desired.

Finally, some of the substances according to the invention also have significant affinity to other pharmacologically interesting receptors, such as for example the serotonin receptor, in particular the 5-HT1 a-receptor, or the dopamine D2-receptor.

In place of a highly selective dopamine D3-receptor binding, depending on the type of illness to be treated, a binding to a further receptor may also be desired.

For example, for the treatment of schizophrenia a compound may be attractive which is a high affinity D3-ligand and at the same time a medium affinity or even high affinity 5-HT1a-receptor ligand. In another embodiment of the invention for the treatment of dyskinesias a compound may be desired which apart from D3-modulatory characteristics also has D2-agonistic, alpha1- and/or 5-HT1a-modulatory characteristics. In other cases, e.g. in the treatment of urinary incontinence, a greater selectivity for the serotonin receptor may in fact be desirable.

The present invention therefore in an excellent manner allows fine tuning of the desired affinity, activity and selectivity in respect of various pharmacologically significant receptors, in particular the dopamine D3-receptors, but also for example in respect of the 5-HT1a-receptor or the D2-receptor.

Forming a further part of the subject-matter of the invention is therefore a pharmaceutical preparation containing one or more of the compounds of general formulae (I) to (IV), or one of the specifically listed compounds as defined above, possibly in the form of a pharmaceutically acceptable salt as well as a pharmaceutically acceptable adjuvant.

The invention also concerns the use of one or more of the compounds of general formulae (I) to (IV), or one of the specifically listed compounds, possibly in the form of a pharmaceutically acceptable salt, for the treatment of the indications mentioned here and the production of a pharmaceutical preparation for the indications mentioned here.

The term “treatment” of an illness covers in this patent application (a) therapy for a pre-existing illness and (b) prevention of an illness that has not developed yet or not yet fully developed, if there is a risk of such an illness occurring.

For the production of pharmaceutical preparations such compounds according to the invention are preferably selected which are high affinity D3-ligands. Particularly preferred is the use of selective or even highly selective D3-ligands.

In another embodiment of the invention compounds are selected which are medium affinity or even high affinity also or in particular for the 5-HT1a-receptor.

The compounds according to the invention have potential in the treatment or prevention of a series of illnesses, which in particular accompany dopamine metabolism or dopaminergic signalling cascade, or possibly serotoninergic signal transmission disorders.

Subject-matter of the invention is therefore the use of a compound according to the invention, as described in this patent application, including the claims and the examples, for the production of a pharmaceutical preparation for the treatment of illnesses which accompany dopamine metabolism and/or dopaminergic signalling cascade disorders.

The subject-matter of the invention is also the use of a compound according to the invention, as described in this patent application, including the claims and the examples, for the production of a pharmaceutical preparation for the treatment of illnesses which accompany serotonin metabolism and/or serotoninergic signal transmission disorders.

Illnesses in whose pathogenesis dopaminergic and/or serotoninergic processes are involved, are in particular illnesses of the central nervous system (CNS). Subject-matter of the invention is therefore the use of a compound according to the invention, as described in this patent application, including the claims and examples, for the production of a pharmaceutical preparation for the treatment of central nervous system illnesses.

The term “central nervous system illnesses” comprises in this patent application both disorders that have their origin in the central nervous system and whose symptoms are predominantly or exclusively noticed in the central nervous system, such as psychoses, depressions or cognitive disorders, and also illnesses which have their origin in the central nervous system, whose symptoms however at least in part are noticed in other target organs, such as extrapyramidal motor disturbances or hyperprolactinemia.

Examples of central nervous system illnesses which can be treated with the compounds according to the invention are:
(1) psychoses and anxiety disorders, including manias, idiopathic psychoses, schizophrenia, compulsive disorders, panic attacks, phobias, eating disorders, aggressive and autoagressive disorders, stereotypies and other personality disorders;
(2) drug dependency, e.g. cocaine, alcohol, opiate and nicotine addiction;
(3) emotional disorders, e.g. depressive disorders, in particular “major depression”, manic-depressive disorders, organically-induced depressions, e.g. in connection with neurodegenerative illnesses such as Parkinson's or Alzheimer's disease;
(4) motor disturbances, including tremors, rigor, dyskinesias, dystonias, such as those associated with Parkinson's disease, parkinsonian syndromes (idiopathically, e.g. in Parkinson-plus-syndrome, or medication-induced, e.g. following L-dopa or neuroleptic treatment), Segawa syndrome, Tourette's syndrome, restless leg syndrome;
(5) sleeping disorders, including dopamine agonist triggered narcolepsy or sleeping disorders associated with Parkinson's disease;
(6) nausea: here dopamine antagonists can be used either alone or in combination with 5-HT3 antagonists;
(7) cognitive disorders and dementias;
(8) hyperprolactinemia; hyperprolactinomia and medically supported ablactation following pregnancy;
(9) glaucoma;
(10) attention deficit hyperactive syndrome (ADHS);
(11) autism, or disorders associated with autism, in particular in the case of compounds with strong serotoninergic active components;
(12) stroke, in particular in the case of compounds with strong serotoninergic active components.

A further therapeutic application that can be mentioned is the treatment and prevention of neurodegenerative diseases, since due to their neuroprotective effect the substances can delay or stop the destruction or loss of neurones as the cause or result of a pathophysiological episode. Such illnesses are for example amyotrophic lateral sclerosis, Alzheimer's disease, Huntington's chorea, epilepsy, Parkinson's disease or synucleopathias, e.g. of the Parkinson-plus-syndrome type.

Apart from the treatment of illnesses which clearly occur or continue with the involvement of the central nervous system, the substances according to the invention can also be used to treat other illnesses which are not clearly or not exclusively associated with the central nervous system. Such illnesses are in particular disorders of the urinary tract, such as sexual dysfunction, in particular male erectile dysfunction and urinary incontinence. For the treatment of urinary incontinence compounds with strong serotoninergic active components are particularly suitable.

Part of the subject-matter of the invention is therefore the use of a compound according to the invention for the production of a pharmaceutical preparation for the treatment of disorders of the urinary tract, in particular of male erectile dysfunction and urinary incontinence.

Illnesses for which the compounds according to the invention are particularly suitable are schizophrenia, depressive disorders, L-dopa- or neuroleptic drug-induced motor disturbances, Parkinson's disease, Segawa syndrome, restless leg syndrome, hyperprolactinemia, hyperprolactinomia, attention deficit hyperactive syndrome (ADHS) and urinary incontinence.

Motor disturbances which are particularly open to therapy with the substances according to the invention are in particular
motor disturbances associated with Parkinson's disease, e.g. rigor, tremor, dystonia and dyskinesia,
Segawa syndrome,
neuroleptic drug-induced (delayed) extrapyramidal motor disturbances, in particular dyskinesia, dystonia and akathisia,
L-dopa-induced extrapyramidal motor disturbances, in particular dyskinesias and dystonias,
restless leg syndrome.

Finally, the pharmaceutical preparations according to the invention, depending on the illness to be treated, can be in the form of a combined preparation for simultaneous or sequential administration.

For example, a sales unit, containing an L-dopa medication for treatment of Parkinson's disease, can also comprise a pharmaceutical composition containing one or more of the compounds according to the invention with, for example, a highly selective, partial agonist dopaminergic and/or serotoninergic profile of action. Here L-dopa and the compound according to the invention can be present in the same pharmaceutical formulation, e.g. a combined tablet, or also in different application units, e.g. in the form of two separate tablets. The two active substances can be administered simultaneously or separately as necessary.

In a combined preparation a sequential administration can, for example, be achieved by the form of administration, e.g. an oral tablet, having two different layers with differing release profiles for the various pharmaceutically active components. It will be clear to the person skilled in the art that in the context of the present invention various forms of administration and application administration schemes are conceivable which are all embodiments of the present invention.

One embodiment of the invention therefore concerns a pharmaceutical preparation containing L-dopa or a neuroleptic drug and a compound according to the invention for simultaneous or timed sequential administration to the patient.

In another embodiment of the invention the sales unit can be a combined preparation or contain two application units, which contain two of the compounds according to the invention with different receptor profiles, e.g. a high affinity, highly selective D3-modulator and a high affinity 5-HT1a-modulator.

Also forming the subject-matter of the invention is a method for treatment of an illness selected from among the illnesses listed in more detail above, through the administration of one or more of the compounds according to the invention, in each case either alone or in combination with other pharmaceutical preparations to a mammal, in need of such treatment, wherein the term “mammal” also and in particular includes humans.

Normally the pharmaceutical preparations according to the invention comprise a pharmaceutical composition which apart from the compounds according to the invention, as described above, contain at least one pharmaceutically acceptable carrier or adjuvant.

It will be clear to the person skilled in the art that the pharmaceutical formulation can be designed differently depending on the envisaged administration route. Thus the pharmaceutical formulation can, for example, be adapted for intravenous, intramuscular, intracutaneous, subcutaneous, oral, buccal, sublingual, nasal, transdermal, inhalative, rectal or intraperitoneal administration.

Appropriate formulations and suitable pharmaceutical carriers or adjuvants, such as fillers, disintegrants, binding agents, lubricants, stabilisers, aromatics, antioxidants, preservatives, dispersions or dissolution agents, buffers or electrolytes, will be known to the person skilled in the art in the area of pharmaceuticals and are for example described in the standard works such as Sucker, Fuchs and Speiser (“Pharmazeutische Technologie”, Deutscher Apotheker Verlag, 1991) and Remington (“The Science and Practice of Pharmacy”, Lippincott, Williams & Wilkins, 2000).

In a preferred embodiment of the invention the pharmaceutical compositions, containing the compounds according to the invention, are administered orally and can, for example, be in the form of capsules, tablets, powders, granulates, coated pills or a liquid.

Here the formulation can be designed as a rapid release form of administration, if fast taking effect is desired. Appropriate oral formulations are, for example, described in EP 0 548 356 or EP 1 126 821.

If, on the other hand, a delayed release is desired, a formulation with delayed active substance release offers itself. Appropriate oral formulations are also known from the prior art.

Alternative pharmaceutical preparations can, for example, be infusion or injection solutions, oils, suppositories, aerosols, sprays, plasters, microcapsules or microparticles.

The compounds of formulae (I) to (IV) are produced using methods that are in part already described in the literature (Bettinetti, L. et al. J. Med. Chem. 2002, 45, 4594-4597). In addition acid derivatives of type (A), which are either synthesised according to the instructions in the literature, generated from commercial preliminary stages or whose production methods are worked out in our laboratories, in the form of their carboxylic acid chlorides or alternatively through the use of special activation reagents such as hydroxybenzotriazole, hydroxyazabenzotriazole, HATU (Kienhöfer, A. Synlett 2001, 1811-1812) or TBTU (Knorr, R. Tetrahedron Lett. 1989, 30, 1927-1930) are activated and with the free base of type (C) converted to the derivatives of formulae (I) and (II):

Production of the compounds according to the invention takes place by reaction of an acid derivative A

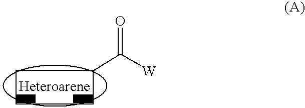

with a free base of general formula C

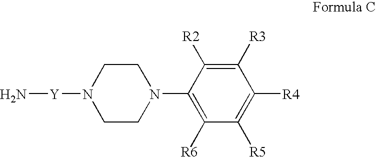

wherein:

W is selected from OH, Cl, Br or a group

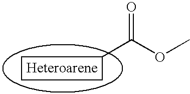

Heteroarene in each case stands for a group which is selected from

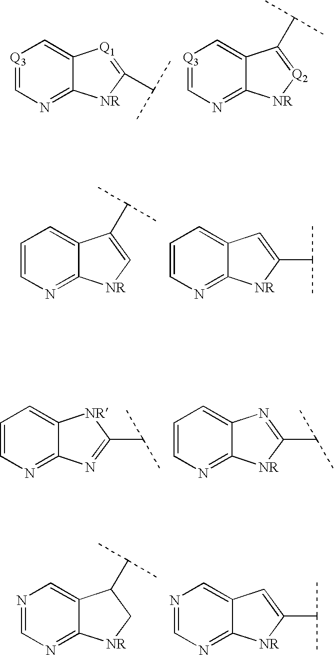

wherein

A, B, Q3 and R in each case have the significance as defined in more detail above in the illustration of the compounds according to the invention;

Q1 and Q2 in each case have the significance as defined above, but do not represent C—X;

the crossed-through bond for the heteroarenes stands for a bond of the —C(O)—W group to a ring-forming C-atom of the 5-membered ring of the heteroarene;

the heteroarene can be substituted once or a number of times with R1, as defined in more detail above;

Y, R2, R3, R4, R5 and R6 in each case have the significance as defined in more detail above,

and wherein in the event that the substituent W is a hydroxy group, the appropriate acid group prior to the conversion with the free base of general formula C is activated by addition of activation reagents such as hydroxybenzotriazole, hydroxyazabenzotriazole, HATU or TBTU.

W is preferably chlorine, bromine or OH particularly preferably chlorine or OH.

### SYNTHESIS OF THE ACID COMPONENTS

### Production of pyrrolopyridine-2-carboxylic Acids

### 1-phenylsulfonylpyrrolo[2,3-b]pyridine-2-carboxylic acid

The production of 1-phenylsulfonylpyrrolo[2,3-b]pyridine-2-carboxylic acid takes place according to the literature (Desarbre, E. Tetrahedron 1997, 3637-3648) via the production of the aldehyde and subsequent oxidation with sodium chlorite.

For this 1.3 ml (2 mmol) 1.6 M n-BuLi are added dropwise to a solution of 0.28 ml (2.0 mmol) diisopropylamine in 3 ml dry THF at −78° C. Then heating is performed to −25° C., 0.258 g (1.0 mmol) 1-phenylsulfonylpyrrolo[2,3-b]pyridine is added dropwise to this solution and agitation is performed for 30 minutes at −25° C. 0.3 ml (4 mmol) DMF, dissolved in 5 ml dry THF, are slowly added dropwise in and agitation is performed for 30 minutes at ambient temperature. Water is added to the reaction solution and it is then neutralised with HCl and absorbed in CH₂Cl₂. Following drying with MgSO₄ the solvent is evaporated. Purification with flash chromatography (SiO₂; petroleum ether-acetic ester:8-2) produces 1-phenylsulfonylpyrrolo[2,3-b]pyridin-2-ylcarbaldehyde.

Yield: 0.123 g (66%).

0.063 g (0.22 mmol) of the aldehyde are dissolved in 5 ml tert.-butylbenzol and 1.2 ml 2-methylbutane are added. A mixture of 0.2 g (0.2 mmol) NaClO₂ and 0.2 g (1.66 mmol) NaH₂PO₄ is added dropwise to this solution over 10 minutes. After 3 hours the solution is evaporated, the residue is washed with hexane and absorbed in water. The aqueous phase is adjusted to pH 3 and extracted with ether. After drying with MgSO₄ the solvent is evaporated and purified by flash chromatography (SiO₂; CH₂Cl₂-MeOH:9-1), which produces 1-phenylsulfonylpyrrolo[2,3-b]pyridine-2-carboxylic acid.

Yield: 89 mg (50%).

M.P.: m/z 302 (M⁺). IR (NaCl): 3323; 1737; 1370; 1179. ¹H NMR (CDCl₃, 360 MHz) δ (ppm): 7.17 (s, 1H, H-3); 7.31 (dd, J=7.8 Hz, J=4.9 Hz, 1H, H-5); 7.54-7.59 (m, 2H, phenylsulfonyl); 7.64-7.69 (m, 1H, phenylsulfonyl); 8.04 (dd, J=7.8 Hz, J=1.6 Hz, 1H, H-4); 8.29-8.31 (m, 2H, phenylsulfonyl); 8.45 (dd, J=4.8 Hz, J=1.6 Hz, 1H, H-6).

### Access to pyrrolopyridine-3-carboxylic Acids

The 1H-pyrrolo[2,3-b]-3-carbaldehyde (0.735 g (5 mmol)) synthesised according to the literature (Verbiscar, A. J., J. Med Chem. 1972, 15,149-152) is dissolved in 15 ml dry DMSO. Then 2.24 g (8 mmol) iodoxybenzoic acid (IBX) are added and N-hydroxysuccinimide added under water bath cooling. The solution is agitated at room temperature for 16 hours and then saturated sodium chloride solution is added, the pH is adjusted with HCl to 3-4 and extraction with diethyl ether takes place. Following drying with MgSO₄ the solvent is evaporated.

Yield: 0.05 g (6%).

MS: m/z 163 ((M+H)⁺).

The production of 1-substituted pyrrolo[2,3-b]pyridine-3-carboxylic acids takes place according to the instructions described in the literature (M. Kato, K. Ito, S. Nishino, H. Yamakuni, H. Takasugi, Chem. Pharm. Bull. 1995, 43,1351-1357; A. Mouaddib, B. Joseph, A. Hasnaoui, J.-Y. Merour Tetrahedron 1999, 40, 5853-5854).

### Access to imidazopyridine-2-carboxylic Acids

The 3H-imidazo[4,5-b]pyridine-2-carboxylic acid was prepared by the conversion of 2,3-diaminopyridine with glycolic acid or lactic acid and subsequent oxidation by means of potassium permanganate (L. Bukowski, M. Janowiec, Z. Zwolska-Kwiek, Z. Andrejczyk Pharmazie, 1999, 54, 651-654).

### Access to pyrrolopyrimidine-6-carboxylic Acid

5-methyl-4-oxo-7-phenyl-4,7-dihydro-3H-pyrrolo[2,3-d]pyrimidine-6-carboxylic acid 5-methyl-4-oxo-7-phenyl-4,7-dihydro-3H-pyrrolo[2,3-d]pyrimidine-6-carboxylic acid ethyl ester (0.050 g, 0.16 mmol; Maybridge, Tintagel/UK, Order code: BTB 090886) are dissolved in 5 ml ethanol. Then 2.5 ml 2n NaOH are added and agitation takes place for 16 hours at ambient temperature. The reaction solution is concentrated in the rotary evaporator and diluted with water, then washed with hexane, adjusted with HCl to pH 3-4 and absorbed in diethyl ether. Following drying with MgSO₄ the solvent is evaporated.

Yield: 0.040 g (90%).

MS: m/z 270 ((M+H)⁺).

### Access to Other Azaindole Carboxylic Acids

Other azaindole carboxylic acids can be prepared according to the synthesis described in the literature (J. H. Musser, T. T. Hudec, K. Bailey, Synth. Comm. 1984, 14, 947-953) of the corresponding pyridine- or pyrimidine-derivates with trialkoxyacetic acid alkyl ester and subsequent saponification. The synthesis of pyrrolopyrimidine-5-carboxylic acid can take place by saponification of the appropriate ester (B. G. Ugarkar et al. J. Med. Chem. 2000, 43, 2883-2893).

### SYNTHESIS OF THE AMINE COMPONENTS

### Production of Type C1 Amines

### 4-phenylpiperazin-1-ylalkylamine, 4-phenylpiperazin-1-ylalkylamine Substituted at the Phenyl Ring

For the production of the type (C1) arylpiperazinylamine commercially available 2-methoxy- or 2,3-dichlorophenylpiperazine, for example, can be alkylated with bromobutylphthalimide in xylol. Subsequent hydrazinolysis of the phthalimide substituted structures provides the type (C1) primary amine. This is explained by way of example in the following reaction diagram:

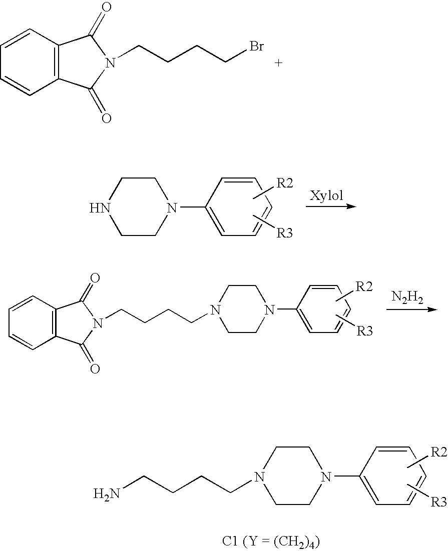

2.3 g (10 mmol) 2,3-dichlorophenylpiperazine (Base) are dissolved in 10 ml xylol and heated to 70° C. Then 1.4 g (5 mmol) 4-bromobutylphthalimide (dissolved in 20 ml xylol) are added dropwise in and the reaction mixture is heated for 24 hours at 125° C. Following cooling of the mixture to 0° C. filtering off is performed and the filtrate is evaporated. The resultant N-(4-(4-(2,3-dichlorophenyl)piperazin-1-yl)butyl)phthalimide is purified by flash chromatography on SiO₂ with ethyl acetate. Yield: 4.0 g (92%).

A solution of 0.45 ml 80% hydrazine hydrate (2.5 eq) in 5 ml ethanol is added dropwise to a suspension of N-(4-(4-(2,3-dichlorophenyl)piperazin-1-yl)butyl)phthalimide in 40 ml ethanol. The mixture is heated for 3 hours with recycling and then cooled to ambient temperature, the resultant solid matter is filtered off, and the ethanolic solution is evaporated in the vacuum. Purification with flash chromatography (CH₂Cl₂-MeOH-Me₂EtN:90-8-2) produces the free base 4-(4-(2,3-dichlorophenyl)piperazin-1-yl)butylamine.

Yield: 0.900 g (60%).

MS: m/z 301 (M⁺), 303 ((M+4)⁺), 305 (M+4)⁺); IR: (NaCl): 3397, 2939, 2817, 1641, 1572, 1500, 1482, 1376, 1240, 1152, 1118, 1023, 917, 791, 749, 698, 661. ¹H NMR (CDCl₃, 360 MHz) δ (ppm): 1.48-1.64 (m, 4H,CH₂—CH₂); 2.44 (t, J=7.6 Hz, 2H, CH₂N); 2.64 (m, 4H, pip); 2.72-2.76 (m, 2H, H₂N—CH₂); 3.07 (m, 4H, pip); 6.93-6.99 (m, 1H, phenyl H-5); 7.11-7.17 (m, 2H, phenyl H-4, phenyl H-6).

### Production of Type C2 Amines

An alternative method of synthesis for obtaining variously substituted type (C2) phenylpiperazinylalkylamines is the reaction of the piperazine with a cyanoalkylhalogenide of appropriate chain length, as explained by way of example in the following reaction diagram:

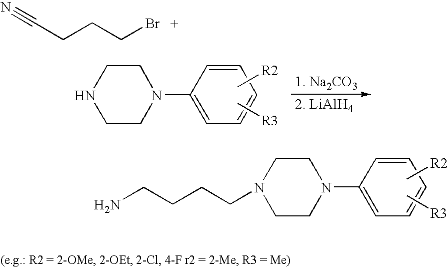

The corresponding 2,3-disubstituted phenylpiperazines are accessible through palladium-catalysed amination of 2,3-substituted halogen aromatic compounds with piperazine:

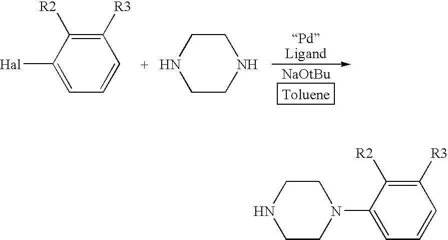

### 4-(4-(3-chloro-2-methoxyphenyl)piperazin-1-yl)butylamine

Thus for the synthesis of 4-(4-(3-chloro-2-methoxyphenyl)piperazin-1-yl)butylamine 1.35 g NaOtBu (14 mmol), 0.024 g Pd(II)acetate (0.5 mol %) and 0.12 g P(OtBu)₃ (2 mol %) are added to 1.7 g (10 mmol) piperazine (base) and dissolved with 1.3 ml dichloroanisol (10 mmol) in 20 ml toluene. After 21 hours of heating to 70° C. the mixture is cooled to ambient temperature, filtered and the filtrate then evaporated in order to obtain 4-(3-chloro-2-methoxyphenyl)piperazine.

Yield: 0.8 g (37%).

0.8 g (3.7 mmol) 4-(3-chloro-2-methoxyphenyl)piperazine and 0.8 g (7.5 mmol) Na₂CO₃ are dissolved in 20 ml acetonitrile, heated for 15 hours with recycling, then cooled to ambient temperature and the solution evaporated in the vacuum. The residue is absorbed in water and the aqueous phase extracted with methylene chloride, this is dried (with MgSO₄) and the solvent is evaporated. Purification with flash chromatography (CHCl₃-EtOAc:1-1) produces 4-(4-(3-chloro-2-methoxyphenyl)piperazin-1yl)butyronitrile.

Yield: 0.4 g (35%).

Then 0.15 g 4-(4-(3-chloro-2-methoxyphenyl)piperazin-1yl)butyronitrile (0.5 mmol) are dissolved in 5 ml dry diethyl ether and cooled to 0° C. Then 1.0 ml LiAlH₄ solution (1 M in diethyl ether) are slowly added dropwise in and agitated for 1 hour at ambient temperature. Following cooling again to 0° C. saturated NaHCO₃ solution is added, filtration is performed through a fritted glass filter with Celite/MgSO₄/Celite and washing is performed with methylene chloride. Evaporation of the filtrate produces 4-(4-(3-chloro-2-methoxyphenyl)piperazin-1-yl)butylamine.

Yield: 0.143 g (96).

MS: m/z 297 (M⁺), 299 ((M+2)⁺), 301 ((M+4)⁺). IR: (NaCl): 3386, 2937, 2821, 1635, 1584, 1540, 1474, 1450, 1251, 1132, 1001, 964, 782, 744, 680, 668. ¹H NMR (CDCl₃, 360 MHz) δ (ppm): 1.60-1.67 (m, 4H, CH₂—CH₂); 2.41-2.45 (m, 2H, H₂N—CH₂); 2.61 (m, 4H, pip); 3.14 (m, 4H, pip); 3.22-3.26 (m, 2H, CH₂N); 3.86 (s, 1H, OCH₃); 6.79-6.82 (m, 1H, phenyl); 6.95 (dd, J=8.0 Hz, J=8.0 Hz, 1H, phenyl H-5); 7.00 (dd, J=1.8 Hz, J=8.0 Hz, 1H, phenyl); 6.95

### 4-(4-(2,3-difluorophenyl)piperazin-1-yl)butylamine

For the production of 4-(4-(2,3-difluorophenyl)piperazin-1-yl)butylamine 0.56 g (5 mmol) piperazine (base) are dissolved with 0.675 g NaOtBu (7 mmol), 0.046 g Pd₂(dba)₃ (0.5 mol %), 0.093 g BINAP (2 mol %) and 0.56 ml (5 mmol) 1-bromine-2,3-difluorobenzol in 20 ml toluene and heated for 18 hours to 115° C. Following cooling of the reaction solution to ambient temperature filtering off is performed and the filtrate is evaporated to obtain 2,3-difluorophenylpiperazine.

Yield: 0.55 g (55%).

The subsequent conversion to 4-(4-(2,3-difluorophenyl)piperazin-1-yl)butylamine takes place analagously to the synthesis described above of type (B2) amines.

Yield: 0.173 g (78% over 2 reaction steps).

MS: m/z 269 (M⁺). IR: (NaCl): 3355, 2939, 2823, 1621, 1585, 1504, 1478, 1269, 1247, 1143, 1007, 774, 714. ¹ H NMR (CDCl₃, 360 MHz) δ (ppm): 1.47-1.60 (m, 4H, CH₂—CH₂); 2.39-2.44 (m, 2H, H₂N—CH₂); 2.61-2.65 (m, 4H, pip); 2.71-2.75 (m, 2H, CH₂N); 3.12-3.15 (m, 4H, pip); 6.67-6.71 (m, 1H, Phenyl); 6.73-6.80 (m, 1H, phenyl); 6.92-6.99 (m, 1H, phenyl).

### Production of Type C3 Amines

### 4-(4-(2,3-dihydrobenzofuran-7-yl)piperazin-1-yl)butylamine, 4-(4-(chroman-8-yl)piperazin-1-yl)butylamine, 4-(4-(2,3,4,5-tetrahydrobenzo[b]oxepin-9-yl)piperazin-1-yl)butylamine

The synthesis takes place to begin with analagously to the literature (Kerrigan, F. Tetrahedron Lett. 1998, 2219-2222) until 2,3-dihydrobenzofuran-7-ylpiperazine has been obtained with a yield of 54% over 4 reaction steps. Then the free base is alkylated analagously to the general conditions for the synthesis of type (C2) amines and the resultant nitrile is reduced to 4-(4-(2,3-dihydrobenzofuran-7-yl)piperazin-1yl)butylamine.

Yield: 0.27 g (86% over 2 reaction steps).

MS: m/z 275 (M⁺). IR: (NaCl): 3359, 2939, 2820, 1609, 1487, 1456, 1254, 1190, 1132, 1012, 942, 870, 755, 661. ¹H NMR (CDCl₃, 360 MHz) δ (ppm): 1.43-1.63 (m, 4H, CH₂—CH₂); 2.34-2.40 (m, 2H, H₂N—CH₂); 2.62 (m, 4H, pip); 2.72-2.74 (m, 2H, O—CH₂—CH₂); 3.15-3.21 (m, 6H, pip, CH₂N); 4.56-4.61 (m, 2H, O—CH₂—CH₂); 6.69-6.71 (m, 1H, phenyl); 6.77-6.86 (m, 2H, phenyl).

The production of 4-(4-(chroman-8-yl)piperazin-1-yl)butylamine takes place analagously to the general conditions for synthesis of type (C3) amines.

Yield: 0.058 g (57% over 2 reaction steps).

MS: m/z289 (M⁺). IR: (NaCl): 3354, 2933, 2870, 2814, 1664,1479, 1461, 1247, 1196, 1024, 870, 737. ¹H NMR (CDCl₃, 360 MHz) δ (ppm): 1.46-1.59 (m, 4H,CH₂—CH₂); 1.96-2.03 (m, 2H, O—CH₂—CH₂—CH₂); 2.39-2.44 (m, 2H, CH₂—N); 2.65 (m, 4H, pip); 2.70-2.74 (m, 2H, O—CH₂—CH₂—CH₂); 2.77-2.80 (m, 2H, CH₂—NH₂); 3.08 (m, 4H, pip); 4.24-4.27 (m, 2H, O—CH₂—CH₂—CH₂); 6.71-6.79 (m, 3H, phenyl).

The production of 4-(4-(2,3,4,5-tetrahydrobenzo[b]oxepin-9-yl)piperazin-1-yl)butylamine takes place analagously to the general conditions for synthesis of type (C3) amines.

Yield: 0.52 g (86%).

MS: m/z 304 [M+H)⁺]. IR: (NaCl): 2933, 2870, 2814, 1666, 1579, 1475, 1450; 1246, 1192, 1038, 785, 733. ¹H NMR (CDCl₃, 360 MHz) δ (ppm): 1.47-1.63 (m, 4H, CH₂—CH₂); 1.68-1.75 (m, 2H, O—CH₂—CH₂—CH₂—CH₂); 1.93-2.00 (m, 4H, H₂O, O—CH₂—CH₂—CH₂—CH₂); 2.41-2.45 (m, 2H, CH₂—N); 2.61-2.65 (m, 4H, pip); 2.73-2.81 (m, 4H, O—CH₂—CH₂—CH₂—CH₂, CH₂—NH₂); 3.10-3.12 (m, 4H, pip); 3.98-4.00 (m, 2H, O—CH₂—CH₂—CH₂—CH₂); 6.77-6.81 (m, 2H, phenyl); 6.88-6.93 (m, 1H, phenyl). ¹³C NMR (CDCl₃, 90 MHz) δ (ppm): 153.5; 144.8; 136.9; 123.9; 123.4; 116.8; 73.3; 58.6; 53.7; 51.0; 42.0; 34.5; 32.5; 31.6; 26.1; 24.3.

### Production of Type C4 Amines

### Trans-4-(4-aminomethylcyclohex-1-ylmethyl)-1-(2-methoxyphenyl)piperazine, trans-4-(4-aminomethylcyclohex-1-ylmethyl)-1-(2,3-dichlorophenyl)piperazine

The synthesis of the amine components with methylcyclohexylmethyl-spacers between amine nitrogen and piperazine is performed as follows:

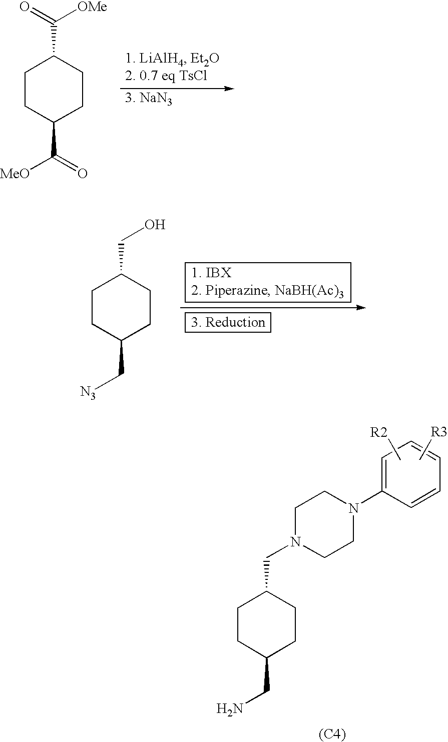

Starting with 1,4-cyclohexylidene dicarboxylic acid dimethyl ester the conversion to 4-azidomethylcyclohex-1-ylmethanol takes place in accordance with the literature (Watanabe, T. Chem. Pharm. Bull. 1995, 43, 529-531). Then oxidation to the aldehyde, reductive amination with the corresponding phenylpiperazines and reduction of the azido group to the primary amine provide the type (C4) amines.

For the synthesis of trans-4-azidomethylcyclohex-1-ylcarbaldehyde 0.10 g (0.6 mmol) trans-4-azidomethylcyclohex-1-ylmethanol are dissolved in 4 ml dry DMSO and following addition of 0.21 g (0.77 mmol) IBX (1-hydroxy-1,2-benziodoxol-3(1H)-one-1-oxide) agitated for 5 hours at ambient temperature. Then diethyl ether and NaHCO₃ solution are added and the organic phase is separated off. This is again washed with NaHCO₃ solution and water and dried over MgSO₄. The solvent is evaporated in the vacuum.

Yield: 75 mg (76%).

MS: m/z 167 (M⁺); IR: (NaCl): 2927, 2856, 2097, 1723, 1452. ¹H NMR (CDCl₃, 360 MHz) δ (ppm): 1.01-1.12 (m, 2H, CH₂—CH₂—CH—CHO); 1.24-1.35 (m, 2H, CH₂—CH₂—CH—CHO); 1.49-1.60 (m, 1H, CH); 1.90-1.95 (m, 2H, CH₂—CH₂—CH—CHO); 2.03-2.07 (m, 2H, CH₂—CH₂—CH—CHO); 2.15-2.24 (m, 1H, CHCHO); 3.18 (d, J=6.8 Hz, 2H, CH₂N₃); 9.63 (d, J=1.4 Hz, 1H, CHO). ¹³C NMR (CDCl₃, 90 MHz) δ (ppm): 204.0, 57.5, 50.0, 41.0, 37.3, 29.8, 29.2, 25.3.

The synthesis of trans-4-(4-azidomethylcyclohexylmethyl)-1-(2-methoxyphenyl)piperazine begins by dissolving 0.39 g (2.3 mmol) trans-4-azidomethylcyclohex-1-ylcarbaldehyde and 0.56 g (2.9 mmol) 2-methoxyphenylpiperazine in 15 ml dichlomethane and the addition of 0.74 g (3.5 mmol) sodium triacetoxyborohydride. After 23 hours of reaction at ambient temperature the mixture is washed with NaHCO₃ solution, and the organic phase is concentrated and purified with flash chromatography (EtOAc-benzene: 1-1).

Yield: 0.78 g (97%).

IR: (NaCl): 2919, 2851, 2812, 2095, 1500, 1450, 1240. ¹H NMR (CDCl₃, 360 MHz) δ (ppm): 0.87-1.05 (m, 4H, CH₂—CH₂); 1.47-1.50 (m, 2H, CH); 1.80-1.91 (m, 4H, CH₂—CH₂); 2.21 (d, J=7.1 Hz, 2H, CH₂Npip); 2.59 (m, 4H, pip); 3.08 (m, 4H, pip); 3.14 (d, J=6.4 Hz, 2H, CH₂N₃); 3.86 (s, 3H, CH₃O); 6.84-7.01 (m, 4H, phenyl).

The synthesis of trans-4-(4-azidomethylcyclohexylmethyl)-1-(2,3-dichlorophenyl)piperazine takes place under identical conditions.

Yield: 0.80 g (85%).

IR: (NaCl): 2930, 2818, 2801, 2096, 1577, 1448. ¹H NMR (CDCl₃, 360 MHz) δ (ppm): 0.87-1.06 (m, 4H, CH₂—CH₂); 1.44-1.59 (m, 2H, CH); 1.81-1.90 (m, 4H, CH₂—CH₂); 2.21 (d, J=7.1 Hz, 2H, CH₂Npip); 2.57 (m, 4H, pip); 3.05 (m, 4H, pip); 3.14 (d, J=6.4 Hz, 2H, CH₂N₃); 6.92-6.97 (m, 1H, phenyl); 7.10-7.16 (m, 4H, phenyl). ¹³C NMR (CDCl₃, 90 MHz) δ (ppm): 151.4, 134.0, 127.5, 127.4, 124.4, 117.5, 65.4, 58.0, 53.8, 51.4, 38.4, 35.0, 31.1, 30.3.

The amine component trans-4-(4-aminomethylcyclohex-1-ylmethyl)-1-(2-methoxyphenyl)piperazine is produced by preparing a solution of 0.40 g (1.2 mmol) trans-4-(4-azidomethylcyclohexylmethyl)-1-(2-methoxyphenyl)piperazine in 10 ml methanol and the addition of 0.10 g Pd/C 10%. The suspension is agitated under an H₂-atmosphere for 23 hours at ambient temperature. Then the solvent is evaporated in the vacuum and purified with flash chromatography (CH₂Cl₂—CH₃OH—NEtMe₂: 90-8-2).

Yield: 0.14 g (39%) (light yellow oil).

MS: 317 m/z (M⁺); IR: (NaCl): 3382, 2912, 2842, 2811, 1500, 1240, 747. ¹H NMR (CDCl₃, 360 MHz) δ (ppm): 0.87-1.05 (m, 4H, CH₂—CH₂); 1.25-1.30 (m, 1H, CH); 1.45-1.56 (m, 1H, CH); 1.81-1.91 (m, 4H, CH₂—CH₂); 2.21 (d, J=7.1 Hz, 2H, H₂N—CH₂); 2.55 (d, J=6.4 Hz, 2H, CH₂Npip); 2.59 (m, 4H, pip); 3.08 (m, 4H, pip); 3.86 (s, 3H, CH₃O); 6.84-7.01 (m, 4H, Phenyl). ¹³C NMR (CDCl₃, 90 MHz) δ (ppm): 152.3, 141.5, 122.7, 120.9, 118.1, 111.1, 65.7, 55.3, 53.9, 50.7, 48.7, 35.3, 31.4, 30.9, 30.4.

For the production of trans-4-(4-aminomethylcyclohex-1-ylmethyl)-1-(2,3-dichlorophenyl)piperazine 25 ml dry THF 1.05 ml LiAlH₄ solution (1 M in THF) is added to a solution of 0.20 g (0.52 mmol) trans-4-(4-azidomethylcyclohexylmethyl)-1-(2,3-dichlorophenyl)piperazine and heated for 8 hours with recycling. The solution is evaporated in the vacuum and purified by flash chromatography (CH₂Cl₂—CH₃OH—NEtMe₂: 90-8-2).

Yield: 0.13 g (36%) (light yellow oil).

MS: 355 m/z (M⁺), 357 ((M+2)⁺), 359 ((M+4)⁺); IR: (NaCl): 3375, 2913, 2843, 2817, 1577, 1448, 778. ¹H NMR (CDCl₃, 360 MHz) δ (ppm): 0.85-0.98 (m, 4H, CH₂—CH₂); 1.19-1.31 (m, 1H, CH); 1.43-1.52 (m, 1H, CH); 1.80-1.88 (m, 4H, CH₂—CH₂); 2.19 (d, J=7.1 Hz, 2H, H₂N—CH₂); 2.53-2.56 (m, 6H, pip, CH₂Npip); 3.06-3.08 (m, 3H, pip); 3.17-3.20 (m, 1H, pip); 6.94-6.96 (m, 1H, Phenyl), 7.10-7.15 (m, 2H, Phenyl).

### SYNTHESIS OF THE EXAMPLE COMPOUNDS

### Example 1

### N-(4-(4-(2-methoxyphenyl)piperazin-1-yl)butyl)-1H-pyrrolo[2,3-b]pyridin-2-ylcarbamide

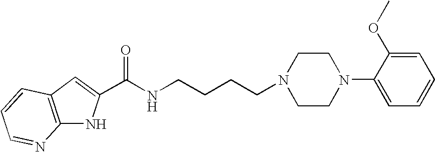

Synthesis analogous to example 2. To obtain the target compound with hydrogen substituted in position 1, starting with the compound in example 2 the phenyl sulfonyl group is separated with KOH in ethanol. For this 2.5 ml 4% KOH and 2.5 ml ethanol are added to 0.04 g (0.07 mmol) N2-(4-(4-(2-methoxyphenyl)piperazin-1-yl)butyl)-1-phenylsulfonyl-1H-pyrrolo[2,3-b]pyridin-2-ylcarboxamide and heated for one hour with recycling. Following cooling of the solution to ambient temperature this is adjusted to pH 9-10 and extracted with methylene chloride, washed with saturated NaHCO₃ solution and saturated NaCl solution and following drying evaporated with MgSO₄. Purification by flash chromatography (SiO₂; petroleum ether-acetic acid 8-2) results in the end product.

Yield: 6 mg (20%) M.P.: 117° C. HPLC/MS m/z 408 (M⁺). IR (NaCl): 3302; 2928; 2813; 1636; 1595; 1498; 1239; 1115; 746. ¹H NMR (CDCl₃, 360 MHz) δ (ppm): 1.64-1.76 (m, 4H, CH₂—CH₂); 2.48 (t, J=7.0 Hz, 2H, CH₂Npip); 2.67 (m, 4H, pip); 3.10 (m, 4H, pip); 3.52-3.58 (m, 2H, CH₂NHCO) 3.85 (s, 3H, OCH₃); 6.68 (br t, J=5.1 Hz, 1H, NHCO); 6.80 (s, 1H, H-3); 6.85 (d, J=7.5 Hz, 1H, Phenyl); 6.90-6.92 (m, 2H, Phenyl); 6.97-7.02 (m, 1H, Phenyl); 7.15 (d, J=4.8 Hz, 1H, H-5); 7.97 (dd, J=8.0 Hz, J=1.6 Hz, 1H, H-4); 8.56 (dd, J=4.6 Hz, J=1.6 Hz, 1H, H-6); 10.99 (s, 1H, H-1).¹³C-NMR (CDCl₃, 90 MHz) δ (ppm): 161.2; 152.3; 148.2; 146.1; 141.2; 131.6; 122.9; 120.9; 118.2; 116.9; 111.2; 118.2; 116.9; 111.2; 100.2; 57.9; 55.3; 53.5; 50.5; 39.6; 27.5, 24.3.

### Example 2

### N-(4-(4-(2-methoxyphenyl)piperazin-1-yl)butyl)-1-phenylsulfonyl-1H-pyrrolo[2,3-b]pyridin-2-ylcarbamide

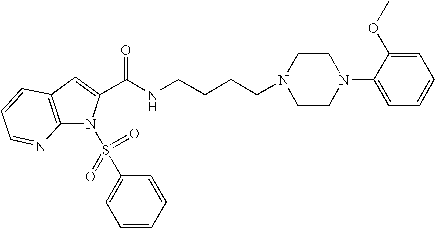

0.036 g (0.12 mmol) of the pyrrolo[2,3-b]pyridine-2-carboxylic acid are dissolved in 4 ml dry methylene chloride and 0.06 ml (0.13 mmol) DIPEA added. In addition at 0° C. 0.065 g (0.13 mmol) of the HATU dissolved in 1 ml DMF are slowly added dropwise in. Then 0.036 g (0.13 mmol) 4-(4-aminobutyl)-1-(2-methoxyphenyl)piperazine are dissolved in methylene chloride and added dropwise in at 0° C. to the reaction solution. After 2 hours the deposit is absorbed in methylene chloride and washed with saturated NaHCO₃ solution and water. After drying with MgSO₄ the solvent is evaporated and purified by flash chromatography (SiO₂; CH₂Cl₂—CH₃OH: 95-5).

Yield: 63 mg (96%).

M.P.: 166° C. MS m/z 548 (M⁺). IR (NaCl): 3398; 2942; 2825; 1655; 1559; 1500; 1375, 1241; 1176; 1027; 752. ¹H NMR (CDCl₃, 360 Mhz) δ (ppm): 1.70-1.84 (m, 4H, CH₂—CH₂); 2.54 (t, J=6.4 Hz, 2H, CH₂Npip); 2.68 (m, 4H, pip); 2.88 (m, 4H, pip); 3.49-3.55 (m, 2H, CH₂NHCO), 3.80 (s, 3H, OCH₃); 6.59-6.62 (m, 1H, phenyl); 6.81-6.85 (m, 2H, H-3, phenyl); 6.98-7.02 (m, 1H, Phenyl); 7.19 (dd, J=4.8 Hz, J=8.0 Hz, 1H, H-5); 7.48-7.60 (m, 4H, phenylsulfonyl, phenyl); 7.82 (dd, J=1.6 Hz, J=7.8 Hz, 1H, phenylsulfonyl); 7-93-7.96 (br.t., J=4.3 Hz, 1H, NHCO); 8.33-8.35 (m, 2H, phenylsulfonyl, H-4); 8.48 (dd, , J=1.6 Hz, J=4.8 Hz, 1H, H-6). ¹³C-NMR (CDCl₃, 90 MHz) δ (ppm): 162.1; 161.8; 152.1; 148.6; 146.2; 140.9; 138.2; 136.1; 134.0; 130.1; 128.9; 128.7; 122.8; 120.9; 120.8; 119.4; 117.9; 111.0; 107.4; 89.3; 57.9; 55.3; 53.2; 50.2; 40.2; 27.2; 24.5.

### Example 3

### N-(4-(4-(2,3-dichlorophenyl)piperazin-1-yl)butyl)-1H-pyrrolo[2,3-b]pyridin-2-ylcarbamide

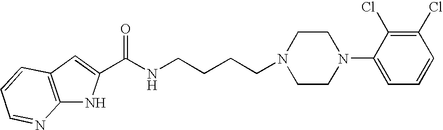

Synthesis starting with the compound of example 4 and under analogous conditions as for example 2. Then separation of the phenylsofonyl group as described for example 1 produces the compound of example 3.

Yield: 12 mg (68% yield).

M.P.: 232° C. MS m/z 445 (M⁺), 447 ((M+2)⁺), 449 ((M+4)⁺). IR (NaCl): 3379; 2924; 2851; 1631; 1557; 1496; 1259; 1028; 758. ¹H NMR (CDCl₃, 360 MHz) δ (ppm): 1.66-1.74 (m, 4H, CH₂—CH₂); 2.49 (t, J=6.7 Hz, 2H, CH₂Npip); 2.66 (m, 4H, pip); 3.07 (m, 4H, pip); 3.52-3.57 (m, 2H, CH₂NHCO); 6.57 (br t, J=4.8 Hz, 1H, NHCO); 6.78 (s, 1H, H-3); 6.91 (dd, J=7.5 Hz, J=2.1 Hz, 1H, phenyl); 7.09-7.17 (m, 3H, phenyl, H-5); 7.97 (dd, J=8.0 Hz, J=1.6 Hz, 1H, H-4); 8.49 (dd, J=4.6 Hz, J=1.4 Hz, 1H, H-6); 10.17 (s, 1H, H-1). ¹³C-NMR (CDCl₃, 90 MHz) δ (ppm): 161.1; 151.1; 147.9; 146.2; 134.0; 131.3; 130.4; 127.5; 127.4; 124.6; 120.0; 118.9; 117.0; 100.2; 57.9; 53.3; 51.2; 39.7; 27.5, 24.3.

### Example 4

### N-(4-(4-(2,3-dichlorophenyl)piperazin-1-yl)butyl)-1-phenylsulfonyl-1H-pyrrolo[2,3-b]pyridin-2-ylcarbamide

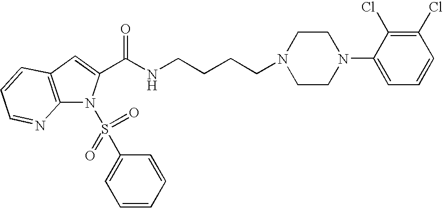

Synthesis analogous to example 2.

Yield: 48 mg (68%).

M.P.: 82° C. MS m/z 586 (M⁺), 588 ((M+2)⁺), 590 ((M+4)⁺). IR (NaCl): 3281; 2937; 2824; 1658; 1578; 1449; 1376; 1239; 1176, 1044; 756. ¹H NMR (CDCl₃, 360 MHz) δ (ppm): 1.65-1.73 (m, 4H, CH₂—CH₂); 2.57 (t, J=6,4 Hz, 2H, CH₂Npip); 2.70 (m, 4H, pip); 2.88 (m, 4H, pip); 3.49-3.55 (m, 2H, CH₂NHCO), 6.55 (dd, J=7.8 Hz, J=1.4 Hz, 1H, Phenyl); 6.74 (s, 1H, H-3); 6.99-7.04 (m, 1H, phenyl); 7.09-7.12 (m, 1H, Phenyl); 7.17 (dd, J=8.0 Hz, J=4.8 Hz, 1H, phenylsulfonyl); 7.48-7.60 (m, 3H, phenylsulfonyl); 7.80 (dd, J=7.8 Hz, J=1.6 Hz, 1H, H-4); 8.01 (br t, J=4.8 Hz, 1H, NHCO); 8.38-8.41 (m, 2H, phenylsulfonyl, H-5); 8.48 (dd, J=4.8 Hz, J=1.8 Hz, 1H, H-6). ¹³C NMR (CDCl₃, 90 MHz) δ (ppm): 161.9; 150.7; 148.7; 146.3; 138.1; 136.7; 134.2; 133.9; 130.2, 128.9; 128.6; 127.4, 124.7; 120.9; 119.6; 118.9; 118.5; 107.9; 95.4; 89.4; 58.0; 53.2; 50.7; 40.0; 27.2; 24.1.

### Example 5

### N-(4-(4-(2-methoxyphenyl)piperazin-1-yl)butyl)-3H-imidazo[4,5-b]pyridin-2-ylcarbamide

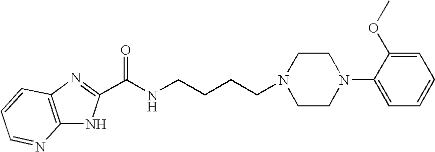

Diisopropylethylamine (220 μL, 1.26 mmol) are added to a solution of the 3H-imidazo[4,5-b]pyridine-2-carboxylic acid (68 mg, 0.42 mmol) in dry DMF (15 mL). TBTU (159 mg, 0.42 mmol; dissolved in 2 mL DMF) are added at 0° C. The ice bath is removed and after 5 minutes of agitation 4-(4-(2-methoxyphenyl)piperazin-1-yl)butylamine (110 mg, 0.42 mmol) dissolved in dichloromethane (2 mL) is added dropwise in. The mixture is agitated for 1 hour at ambient temperature. Then saturated NaHCO₃ solution and dichloromethane are added, the phases are separated and the aqueous phase is extracted with dichloromethane (2×). The combined organic phases are washed with saturated NaCl solution, dried over sodium sulphate and concentrated. Following flash chromatography (SiO₂, dichloromethane/methanol 9:1) the product is obtained (62 mg, 36%) as colourless solid matter.

MS (APCI) m/z 409 [(M+H)⁺]; ¹H-NMR (CDCl₃, 200 MHz) δ (ppm): 1.64-1.66 (m, 4H), 2.45-2.48 (m, 2H), 2.67-2.71 (m, 4H), 3.03-3.09 (m, 4H), 3.42-3.45 (m, 2H), 3.79 (s, 3H), 6.77-6.95 (m, 4H), 7.22-7.29 (m, 1H), 7.94-7.99 (m, 1H), 8.43-8.46 (m, 1H).

¹³C-NMR (CDCl₃, 50 MHz) δ (ppm): 23.4, 27.1, 39.2, 49.9, 53.0, 55.2, 57.9, 111.1, 118.2, 119.4, 120.9, 123.2, 140.5, 145.8, 146.6, 152.1, 158.7.

### Example 6

### N-(4-(4-(2,3-dichlorophenyl)piperazin-1-yl)butyl)-3H-imidazo[4,5-b]pyridin-2-ylcarbamide

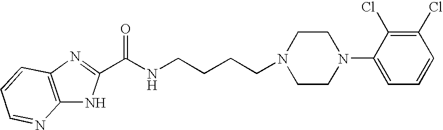

Synthesis analogous to example 5.

Yield: 29 mg (21%)

MS (APCI) m/z 447 [(M+H)⁺], 328 (100%); ¹H-NMR (CDCl₃, 200 MHz) δ (ppm): 1.69-1.71 (m, 4H), 2.50-2.60 (m, 2H), 2.70-2.76 (m, 4H), 3.05-3.10 (m, 4H), 3.45-3.55 (m, 2H), 6.95-7.00 (m, 1H), 7.14-7.16 (m, 2H), 7.24-7.36 (m 1H), 8.00-8.05 (m, 1H), 8.45-8.48 (m, 1H); ¹³C-NMR (CDCl₃, 50 MHz) δ (ppm): 23.1, 26.8, 39.0, 49.5, 50.2, 52.7, 57.6, 118.1, 118.4, 119.4, 120.9, 124.5, 127.0, 127.2, 133.6, 142.9, 143.7, 145.4, 146.5, 147.4, 150.4, 150.7, 158.6.

### Example 7

### N-(4-(4-(2-chlorophenyl)piperazin-1-yl)butyl)-3H-imidazo[4,5-b]pyridin-2-ylcarbamide

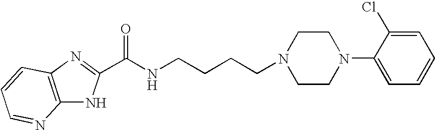

Instructions analogous to example 5.

Yield: 73 mg (35%)

MS (APCI) m/z 413 [(M+H], 294; ¹H-NMR (CDCl₃, 200 MHz) δ (ppm): 1.65-1.72 (m, 4H), 2.46-2.53 (m, 2H), 2.67-2.78 (m, 4H), 3.08-3.12 (m, 4H), 3.53-3.59 (m, 2H), 6.89-6.99 (m, 2H), 7.02-7.37 (m, 3H), 7.95 (wide s, 1H), 8.07-8.11 (m, 2H), 8.74-8.76 (m, 1H); ¹³C-NMR (CDCl₃, 50 MHz) δ (ppm): 24.1, 27.3, 39.5, 40.9, 53.3, 57.8, 113.2, 119.2, 120.3, 123.6, 127.5, 128.7, 130.5, 145.9, 146.8, 149.1, 158.8.

### Example 8

### N-(4-(4-(2,3-dimethylphenyl)piperazin-1-yl)butyl)-3H-imidazo[4,5-b]pyridin-2-ylcarbamide

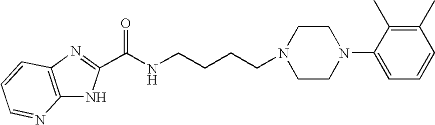

Instructions analogous to example 5.

Yield: 30 mg (21%)

MS (APCI): m/z 407 [(M+H)⁺], 288; ¹H-NMR (CDCl₃, 200 MHz) δ (ppm): 1.62-1.64 (m, 4H), 2.13 (s, 3H), 2.16 (s, 3H), 2.46-2.50 (m, 2H), 2.63-2.80 (m, 4H), 2.83-2.88 (m, 4H), 3.40-3.44 (m, 2H), 6.78-6.83 (m, 2H), 6.93-6.96 (m, 1H), 7.20-7.27 (m, 1H), 7.93-7.96 (m, 1H), 8.40-8.44 (m, 1H); ¹³C-NMR (CDCl₃, 50 MHz) δ (ppm): 13.8, 20.5, 23.6, 27.2, 39.4, 51.5, 53.6, 58.1, 116.6, 119.6, 125.2, 125.9, 131.2, 138.0, 145.9, 146.7, 151.1, 158.9.

### Example 9

### N-(4-(4-(4-fluorophenyl)piperazin-1-yl)butyl)-3H-imidazo[4,5-b]pyridin-2-ylcarbamide

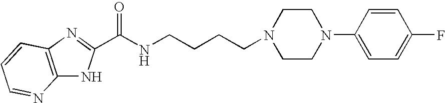

Instructions analogous to example 5.

Yield: 42 mg (34%)

MS (APCI) m/z 397 [(M+H)⁺], 278; ¹H-NMR (CDCl₃, 200 MHz) δ (ppm): 1.65-1.75 (m, 4H), 2.45-2.49 (m, 2H), 2.61-2.77 (m, 4H), 3.12-3.28 (m, 4H), 3.55-3.58 (m, 2H), 6.79-6.95 (m, 4H), 7.29-7.36 (m, 1H), 7.95-8.08 (m, 2H), 8.71-8.75 (m, 1H); ¹³C-NMR (CDCl₃, 50 MHz) δ (ppm): 24.1, 27.3, 39.5, 49.9, 53.1, 57.8, 115.4 (J=22 Hz), 117.7 (J=8 Hz), 119.2, 145.9, 146.8, 147.8, 147.9, 157.1 (J=239 Hz), 158.8.

### Example 10

### N-(4-(4-(2,3-dichlorophenyl)piperazin-1-yl)butyl)-1H-pyrrolo[2,3-b]pyridin-3-ylcarbamide

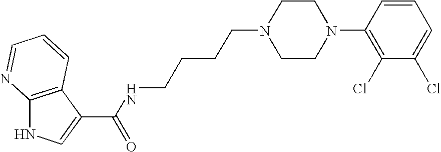

Instructions analogous to example 5.

Yield: 25 mg (23%).

MS: m/z 447 (M⁺); ¹H NMR (CDCl₃, 360 MHz) δ (ppm): 1.68-1.74 (m, 4H, CH₂—CH₂); 2.53 (t, J=6.9 Hz, 2H, CH₂N); 2.66-2.71 (m, 4H, pip); 3.04-3.08 (m, 4H, pip); 3.51-3.56 (m, 2H, CH₂NHCO); 6.36 (br t, J=5.4 Hz, 1H, NHCO); 6.89 (dd, J=1.8 Hz, J=7.7 Hz, 1H, H-arom); 7.08-7.16 (m, 2H, H-arom); 7.21 (dd, J=4.8 Hz, J=7.9 Hz, 1H, H-4); 7.88 (s, 1H, H-2); 8.35 (dd, J=1.6 Hz, J=4.8 Hz, 1H, H-6); 8.43 (dd, J=1.5 Hz, J=8.1 Hz, 1H, H-5); 11.07 (br s, 1H, H-1).

### Example 11

### N-(4-(4-(2-methoxyphenyl)piperazin-1-yl)butyl)-1-methyl-1H-pyrrolo[2,3-b]pyridin-3-ylcarbamide

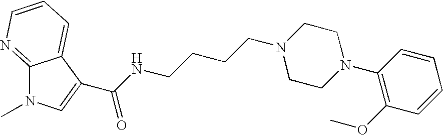

Instructions analogous to example 5.

Yield: 24 mg (26%).

MS (APCI) m/z 422 [(M+H)⁺]; ¹H-NMR (CDCl₃, 200 MHz) δ (ppm): 1.65-1.77 (m, 4H), 2.58-2.66 (m, 2H), 2.81-2.83 (m, 4H), 3.10-3.14 (m, 4H), 3.47-3.53 (m, 2H), 3.82 (s, 3H), 3.84 (s, 3H), 6.62 (wide s, 1H), 6.80-6.99 (m, 4H), 7.11-7.18 (m, 1H), 7.81 (1, 1H), 8.32-8.40 (m, 2H); ¹³C-NMR (CDCl₃, 50 MHz) δ (ppm): 23.1, 27.2, 31.6, 38.7, 49.5, 52.9, 55.3, 57.5, 109.2, 111.1, 112.7, 117.2, 118.2, 118.5, 121.0, 123.4, 124.8, 129.3, 131.1, 140.4, 143.7, 147.8, 152.1, 164.7, 176.3.

### Example 12

### N-(4-(4-(2,3-dichlorophenyl)piperazin-1-yl)butyl)-1-phenylsulfonyl-1H-pyrrolo[2,3-b]pyridin-3-ylcarbamide

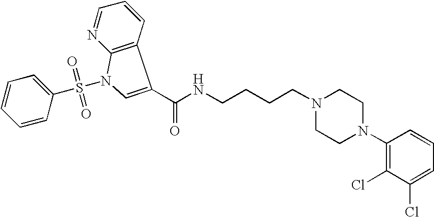

Instructions analogous to example 5.

Yield: 25 mg (23%).

MS (APCI) m/z 586 [(M+H)⁺]; ¹³C-NMR (CDCl₃, 50 MHz) δ (ppm): 25.1, 28.1, 40.2, 51.9, 54.0, 59.1, 119.3, 120.6, 125.4, 126.8, 128.2, 129.0, 129.9, 131.7, 135.3, 146.6.

### Example 13

### N-(4-(4-(2-methoxyphenyl)piperazin-1-yl)butyl)-1-phenylsulfonyl-1H-pyrrolo[2,3-b]pyridin-3-ylcarbamide

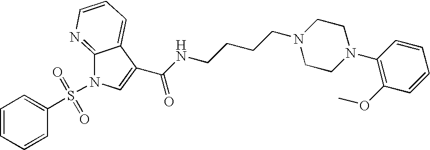

Instructions analogous to example 5.

Yield: 32 mg (27%)

MS (APCI) m/z 548 [(M+H)⁺]; ¹H-NMR (CDCl₃, 200 MHz) δ (ppm): 1.66-1.70 (m, 4H), 2.46-2.49 (m, 2H), 2.67-2.71 (m, 4H), 3.06-3.10 (m, 4H), 3.45-3.48 (m, 2H), 3.83 (s, 3H), 3.84 (s, 3H), 6.81-7.02 (m, 5H), 7.20-7.27 (m, 1H), 7.40-7.60 (m, 3H), 8.15-8.21 (m, 3H), 8.40-8.44 (m, 2H); ¹³C-NMR (CDCl₃, 50 MHz) δ (ppm): 24.3, 27.5, 39.4, 50.4, 53.4, 55.4, 58.0, 11.1, 114.8, 118.3, 119.9, 121.1, 123.1, 126.4, 128.3, 129.2, 131.0, 134.5, 137.7, 141.0, 145.9, 147.0, 152.2, 162.6, 163.1.

### Example 14

### N-(4-(4-(2-ethoxyphenyl)piperazin-1-yl)butyl)-1-phenylsulfonyl-1H-pyrrolo[2,3-b]pyridin-3-ylcarbamide

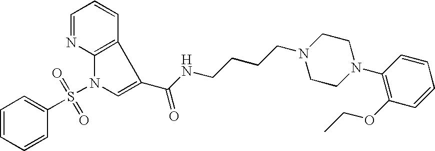

Instructions analogous to example 5.

Yield: 24 mg (23%).

MS (APCI): m/z 562 [(M+H)⁺]; ¹H-NMR (CDCl₃, 200 MHz) δ (ppm): 1.41 (t, J=7.0 Hz, 1.65-1.69 (m, 4H), 2.46-2.49 (m, 2H), 2.60-2.70 (m, 4 H), 3.05-3.15 (m, 4H), 3.43-3.46 (m, 2H), 4.02 (q, J=7.0 Hz), 6.78-6.95 (m, 4H), 7.18-7.27 (m, 2H), 7.39-7.55 (m, 3H), 8.14-8.18 (m, 2H), 8.26 (s, 1H), 8.38-8.46 (m, 2H); ¹³C-NMR (CDCl₃, 50 MHz) δ (ppm): 14.8, 23.9, 27.2, 39.1, 50.0, 53.3, 57.8, 63.5, 112.3, 114.6, 116.1, 119.8, 120.9, 121.1, 122.9, 126.6, 128.1, 129.1, 131.0, 134.4, 137.6, 140.8, 145.6, 146.9, 151.4, 163.2.

### Example 15

### N-(4-(4-(2,3-dimethylphenyl)piperazin-1-yl)butyl)-1-phenylsulfonyl-1H-pyrrolo[2,3-b]pyridin-3-ylcarbamide

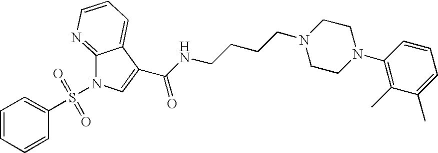

Synthesis analogous to example 5.

Yield: 20 mg (25%).

MS (APCI): m/z 546 ([M+H]⁺); ¹H NMR (CDCl₃, 200 MHz) δ (ppm): 1.63-1.75 (m, 4H), 2.19 (s, 3H), 2.24 (s, 3H), 2.47-2.73 (m, 6H), 2.86-2.92 (m, 4H), 3.42-3.53 (m, 2H), 6.83-6.92 (m, 2H), 7.01-7.09 (m, 1H), 7.21-7.28 (m, 1H), 7.41-7.61 (m, 4H), 8.15-8.22 (m, 3H), 8.39-8.45 (m, 2H).

### Example 16

### N-(4-(4-(2,3-dichlorophenyl)piperazin-1-yl)butyl)-5-methyl-4-hydroxy-7-phenyl-7H-pyrrolo[2,3-d]pyrimidin-6-ylcarbamide or oxo-tautomer

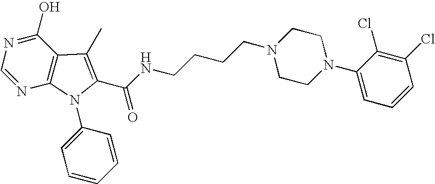

Synthesis analogous to example 5.

Yield: 10 mg (45%).

MS: m/z 554 ([M+H]⁺); ¹H NMR (CDCl₃, 360 MHz) δ (ppm): 1.43-1.53 (m, 4H, CH₂—CH₂); 2.44 (t, J=6.7 Hz, 2H, CH₂N); 2.62-2.72 (m, 4H, pip); 2.68 (s, 3H, CH₃); 2.98-3.07 (m, 4H, pip); 3.28-3.32 (m, 2H, CH₂NHCO); 6.31 (br t, J=5.4 Hz, 1H, NHCO); 6.86 (dd, J=2.4 Hz, J=7.2 Hz, 1H, H-arom); 7.10-7.17 (m, 2H, H-arom); 7.39-7.54 (m, 5H, Phenyl); 7.86 (s, 1H, H-2); 11.21 (br s, 1H, H-3).

### SYNTHESIS OF OTHER POSSIBLE EXAMPLE COMPOUNDS

### Example 17

### N-(4-(4-(2,3-dihydrobenzofuran-7-yl)piperazin-1-yl)butyl)-1H-pyrrolo[2,3-b]pyridin-2-ylcarbamide

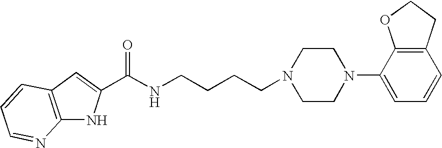

The coupling of the acid component and the amine component can take place analogously to example 2, wherein the acid component and the amine component C3 are produced as described in more detail above.

### Example 18

### N-(4-(4-(2,3-dihydrobenzofuran-7-yl)piperazin-1-yl)butyl)-1-phenylsulfonyl-1H-pyrrolo[2,3-b]pyridin-2-ylcarbamide

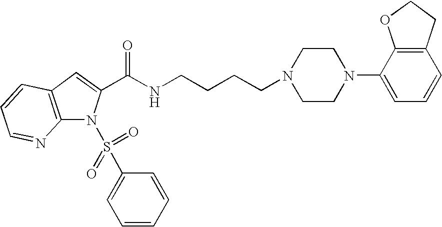

The synthesis can take place analagously to the production of example 17.

### Example 19

### N-(4-(4-(chroman-8-yl)piperazin-1-yl)butyl)-1H-pyrrolo[2,3-b]pyridin-2-ylcarbamide

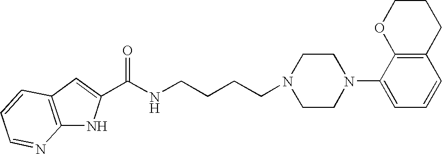

The synthesis can take place analagously to the production of example 17.

### Example 20

### N-(4-(4-(chroman-8-yl)piperazin-1-yl)butyl)-1-phenylsulfonyl-1H-pyrrolo[2,3-b]pyridin-2-ylcarbamide

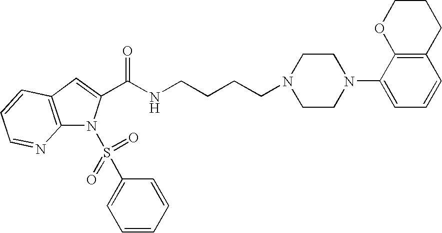

The synthesis can take place analagously to the production of example 17.

### Example 21

### N-(4-(4-(2,3,4,5-tetrahydrobenzo[b]oxepin-9-yl)piperazin-1-yl)butyl)-1H-pyrrolo[2,3-b]pyridin-2-ylcarbamide

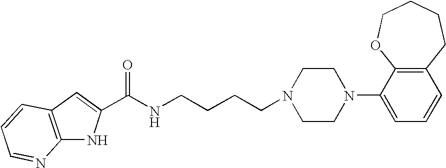

The synthesis can take place analagously to the production of example 17.

### Example 22

### N-(4-(4-(2,3,4,5-tetrahydrobenzo[b]oxepin-9-yl)piperazin-1-yl)butyl)-1-phenylsulfonyl-1H-pyrrolo[2,3-b]pyridin-2-ylcarbamide

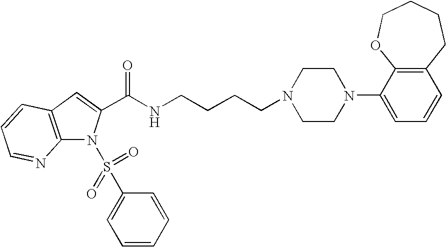

The synthesis can take place analagously to the production of example 17.

### Example 23

### N-(4-(4-(2,3-dihydrobenzofuran-7-yl)piperazin-1-yl)butyl)-1H-pyrrolo[2,3-b]pyridin-3-ylcarbamide

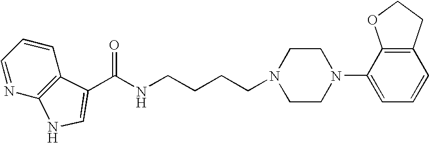

The synthesis can take place analagously to the production of example 17.

### Example 24

### N-(4-(4-(2,3-dihydrobenzofuran-7-yl)piperazin-1-yl)butyl)-1-phenylsulfonyl-1H-pyrrolo[2,3-b]pyridin-3-ylcarbamide

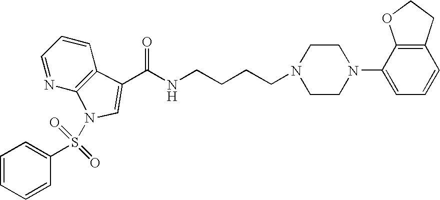

The synthesis can take place analagously to the production of example 17.

### Example 25

### N-(4-(4-(chroman-8-yl)piperazin-1-yl)butyl)-1H-pyrrolo[2,3-b]pyridin-3-ylcarbamide

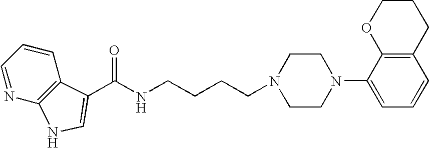

The synthesis can take place analagously to the production of example 17.

### Example 26

### N-(4-(4-(chroman-8-yl)piperazin-1-yl)butyl)-1-phenylsulfonyl-1H-pyrrolo[2,3-b]pyridin-3-ylcarbamide

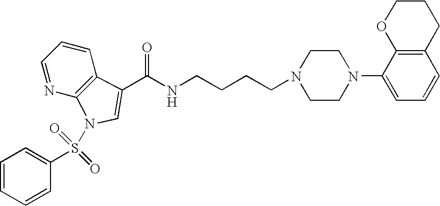

The synthesis can take place analagously to the production of example 17.

### Example 27

### N-(4-(4-(2,3,4,5-tetrahydrobenzo[b]oxepin-9-yl)piperazin-1-yl)butyl)-1H-pyrrolo[2,3-b]pyridin-3-ylcarbamide

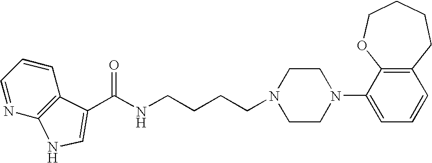

The synthesis can take place analagously to the production of example 17.

### Example 28

### N-(4-(4-(2,3,4,5-tetrahydrobenzo[b]oxepin-9-yl)piperazin-1-yl)butyl)-1-phenylsulfonyl-1H-pyrrolo[2,3-b]pyridin-3-ylcarbamide

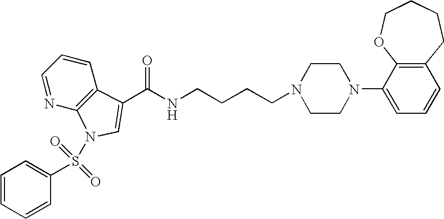

The synthesis can take place analagously to the production of example 17.

### Example 29

### N-(4-(4-(2,3-dihydrobenzofuran-7-yl)piperazin-1-yl)butyl)-3H-imidazo[4,5-b]pyridin-2-ylcarbamide

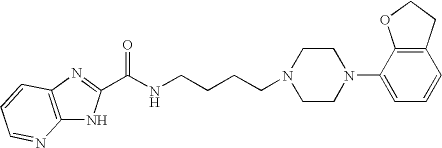

The coupling of the acid component and the amine component can take place analogously to example 5, wherein the acid component and the amine component C3 are produced as described in more detail above.

### Example 30

### N-(4-(4-(chroman-8-yl)piperazin-1-yl)butyl)-3H-imidazo[4,5-b]pyridin-2-ylcarbamide

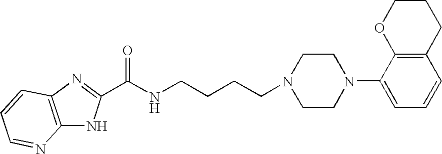

The synthesis can take place analagously to the production of example 29.

### Example 31

### N-(4-(4-(2,3,4,5-tetrahydrobenzo[b]oxepin-9-yl)piperazin-1-yl)butyl)-3H-imidazo[4,5-b]pyridin-2-ylcarbamide

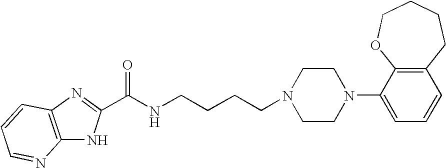

The synthesis can take place analagously to the production of example 29.

### Example 32

### N-(4-(4-(2,3-dihydrobenzofuran-7-yl)piperazin-1-yl)butyl)-5-methyl-4-hydroxy-7-phenyl-7H-pyrrolo[2,3-d]pyrimidin-6-ylcarbamide or oxo-tautomer

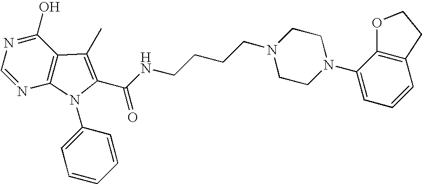

The synthesis can take place analagously to the production of example 29.

### Example 33

### N-(4-(4-(chroman-8-yl)piperazin-1-yl)butyl)-5-methyl-4-hydroxy-7-phenyl-7H-pyrrolo[2,3-d]pyrimidin-6-ylcarbamide or oxo-tautomer

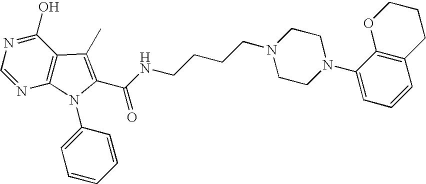

The synthesis can take place analagously to the production of example 29.

### Example 34

### N-(4-(4-(2,3,4,5-tetrahydrobenzo[b]oxepin-9-yl)piperazin-1-yl)butyl)-5-methyl-4-hydroxy-7-phenyl-7H-pyrrolo[2,3-d]pyrimidin-6-ylcarbamide or oxo-tautomer.

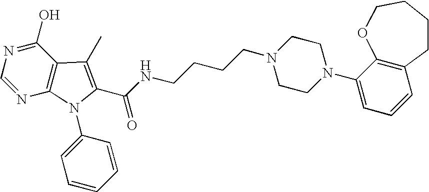

The synthesis can take place analagously to the production of example 29.

### Example 35

### N-(4-(4-(2-methoxyphenyl)piperazin-1-yl)butyl)-5-methyl-4-hydroxy-7-phenyl-7H-pyrrolo[2,3-d]pyrimidin-6-ylcarbamide or oxo-tautomer

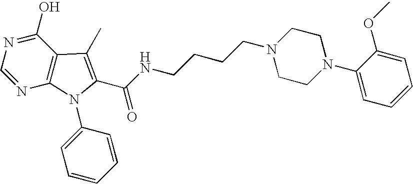

The synthesis can take place analagously to the production of example 5.

### BIOLOGICAL ACTIVITY

The biological activities of the compounds according to the invention were determined in radioligand bonding investigations. All radioligand experiments were performed according to methods described by us (Hübner, H. et al. J. Med. Chem. 2000, 43, 756-762). For the measurement of the affinities to the receptors of the D2-family membrane homogenates of Chinese hamster ovary cells (CHO cells) were used, which stably express the human D2long-, the human D2short- (Hayes, G. et al. Mol. Endocrinol. 1992, 6, 920-926), the human D3- (Sokoloff, P. et al. Eur. J. Pharmacol. 1992, 225, 331-337) or the human D4.4-receptor sub-type, (Asghari, V. J. Neurochem. 1995, 65,1157-1165) respectively. Basically the binding assays took place by incubation of the receptor homogenates with the radioligand [³H]spiperone and the compounds under investigation in various concentrations. Determination of the affinities to the D1-receptor took place with native membrane homogenates, obtained from porcine striatum, and the D1-selective radioligands [³H]SCH 23390.

Measurement of the bonding strengths of the compounds to the serotonin-receptor subtypes 5-HT1A and 5-HT2 was carried out according to methods described by us (Heindl, C. et al. Tetrahedron: Asymmetry 2003, 14, 3141-3152). For this we incubated porcine cortex-membrane preparations with the radioligands [³H]8-OH-DPAT (for 5-HT1A) or [³H]ketanserin (5-HT2) and the compounds in various concentrations. In the same way the affinity of the test compounds to the porcine α1-receptor was investigated, wherein porcine cortex-membrane preparations and the α1-selective radioligand [³H]prazosin were used.

All compounds investigated in the dopamine receptor-binding assay demonstrated good to very good affinities to the dopamine receptors with a clear binding preference to D2 and D3 subtypes. Pyrrolopyridines exhibit a particularly high D3 affinity if they carry a distance of 4-5 carbon atoms between the amide nitrogen and the nitrogen of the piperazine component. There is always a clear selectivity to the D3 receptor here, which for all the compounds tested was associated with Ki-values of between 0.1 and approximately 10 nM. (Table 1)

TABLE 1
Bonding data and selectivity specimens of the compounds of formula I
and II for the dopamine-receptors porcinD1, humanD2long,
humanD2short, humanD3 and humanD4.4^{a}

| | | | | | | D3-selectivity | | |
|---|---|---|---|---|---|---|---|---|
| | Ki-value in [nM]^{b} | | | | | D2long/ | D2short/ | D4.4/ |
| Compound | D1 | D2long | D2short | D3 | D4.4 | D3 | D3 | D3 |
| Example 1 | 1500 | 58 | 42 | 0.82 | 32 | 71 | 51 | 39 |
| Example 2 | 1300 | 180 | 110 | 9.3 | 130 | 19 | 12 | 14 |
| Example 3 | 440 | 19 | 6.5 | 0.13 | 42 | 150 | 34 | 320 |
| Example 4 | 680 | 68 | 39 | 0.80 | 110 | 85 | 49 | 140 |

^{a}determined for D2long, D2short, D3 and D4.4 with the radioligand [³H]spiperon and for D1 with [³H]SCH 23390;
^{b}Average values from 2-6 individual experiments in each case performed in triplicate

Investigations to determine the intrinsic activity of the example compounds were carried out in a mitogenesis assay in accordance with the literature (Hübner, H. et al. J. Med. Chem. 2000, 43, 4563-4569; Bettinetti, L. et al. J. Med. Chem. 2002, 45, 4594-4597). Here various concentrations of the compound under investigation were incubated with D3 receptor-expressing cells and then the receptor-mediated stimulation of the mitogenesis rate was measured by incorporation of the radioactive marker [³H]thymidine. Agonistic, partial agonistic or antagonistic effects were determined in comparison with the effect of the full agonist quinpirol. (Table 2)

TABLE 2
Results of the mitogenesis experiments with the embodiment
examples at the dopamine-D3-receptor for determination of the
intrinsic activity^{a}

| | Number of | | |
|---|---|---|---|
| | individual | | |
| Compounds | measurements | EC₅₀ value [nM]^{b} | agonistic activity [%]^{c} |
| Example 1 | 12 | 0.96 | 14 |
| Example 2 | 6 | 7.9 | 12 |
| Example 3 | 12 | 1.3 | 41 |
| Example 4 | 6 | 2.1 | 16 |
| Quinpirol | 5 | 3.2 | 100 |

^{a}dose-dependent incorporation of the radiomarker [³H]thymidine as a measure of the stimulation of the mitogenesis rate measured at seven different concentrations in quadruplicate.
^{b}EC₅₀ value of the dose-effect curve derived from the average values of all the individual trials (n)
^{c}agonistic activities in [%] with reference to the maximum effect of the full agonist Quinpirol

In this test for the compound under investigation various intrinsic effects at the D3-receptor were measured. Thus compounds 1, 2 and 4 demonstrate a stimulation of the receptor in the range 10% -20% and can rather be classified as weakly partially agonistic, whereas example compound 3 with an intrinsic activity of 41% can be classified as a partial agonist.

The investigation of the affinities to the serotonin receptor subtypes 5-HT1A and 5-HT2 and to the α-1 adrenergic receptor are described in Table 3.

The binding strength to the serotonin and alpha-1 receptors with affinities in the range up to 25 nM can also be characterised as very strong, wherein three of the four examples tested demonstrated a clear selectivity to the 5-HT1A receptor compared with the 5-HT2 subtype.

TABLE 3
Results of the bonding investigations with substances according to
formula I and II to the serotonin receptors porcin5-HT1A, porcin5-HT2
and to the porcine adrenergic receptor subtype α1^{a}

| Com- | Ki-values in [nM]^{b} | | | D3-selectivity | | |
|---|---|---|---|---|---|---|
| pounds | 5-HT1A | 5-HT2 | α1 | 5-HT1A/D3 | 5-HT2/D3 | α1/D3 |
| Example 1 | 23 | 340 | 1.3 | 28 | 1200 | 1.6 |
| Example 2 | 14 | 1100 | 4.6 | 1.5 | 120 | 0.49 |
| Example 3 | 24 | 63 | 3.6 | 180 | 480 | 28 |
| Example 4 | 16 | 200 | 21 | 20 | 250 | 26 |

^{a}determined for 5-HT1A with the radioligand [³H]8-OH-DPAT, for 5-HT2 with [³H]ketanserin and for α1 with [³H]prazosin
^{b}average values from 2 individual experiments in each case carried out in triplicate

1. A compound of general formula I, 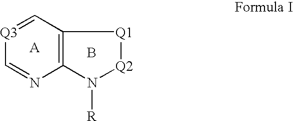
a physiologically acceptable salt thereof, or an enantiomer, diastereomer, or tautomer thereof, wherein
A is an aromatic 6-membered ring wherein the ring-forming C-atoms independently and optionally carry R1;
B is an aromatic 5-membered ring which carries exactly one X group;
Q1 is N, N—R′, S, O, CH, C—R1 or C—X;
Q2 is CH, C—R1 or C—X, wherein either Q1 or Q2 is a C—X group;
Q3 is N, CH or C—R1;
R1 is in each occurrence independently selected from hydroxy, alkyl, alkyloxy, alkylthio, alkenyl, alkynyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino;
R′ is selected from hydrogen, alkyl, phenyl, phenylalkyl, alkylcarbonyl, phenylcarbonyl, phenylalkylcarbonyl and phenylsulfonyl;
R is absent if Q1 is N—R′, S or O; or R is selected from hydrogen, alkyl, phenyl, phenylalkyl, alkylcarbonyl, phenylcarbonyl, phenylalkylcarbonyl and phenylsulfonyl if Q1 is N, CH, C—R1 or C—X, and
X is a group of general formula X1 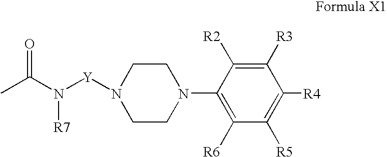
wherein:
Y is an unbranched, saturated or unsaturated hydrocarbon chain with 2-5 carbon atoms or a chain —(CH₂)ₒ-Z-(CH₂)ₚ,
wherein Z is selected from cyclopentyl, cyclohexyl and cycloheptyl residues, o and p independently have the value of 0, 1, 2 or 3, and the sum of o and p is at most 3;
R2, R3, R4, R5 and R6 are independently selected from hydrogen, hydroxy, alkyl, alkyloxy, alkylthio, alkenyl, alkynyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein in addition two vicinal residues of R2, R3, R4, R5 and R6 optionally form an oxygen-containing 5-, 6- or 7-membered ring together with the C-atoms of the phenyl ring to which they are bonded; and
R7 is hydrogen or alkyl.

2. The compound according to claim 1,
wherein
Q1 is N, N—R′, CH, C—R1 or C—X;
R1 is independently selected from hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl, in which the phenyl is unsubstituted or substituted with fluorine, chlorine or bromine and/or with one or more methoxy groups, and the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl in which the alkyl is unsubstituted or hydroxy substituted; —C(O)-phenyl in which the phenyl is unsubstituted or substituted with fluorine, chlorine or bromine and/or with one or more methoxy groups; —C(O)—(C1-C6)alkyl-phenyl in which the phenyl is unsubstituted or substituted with fluorine, chlorine or bromine and/or with one or more methoxy groups, and the C1-C6 alkyl is unsubstituted or hydroxy substituted; C1-C6 alkyloxycarbonyl in which the alkyl is unsubstituted or hydroxy substituted; phenyl(C1-C6)alkyloxycarbonyl in which the phenyl is unsubstituted or substituted with fluorine, chlorine or bromine and/or substituted with one or more methoxy groups and the C1-C6 alkyl is unsubstituted or hydroxy substituted; C1-C6 alkylaminosulfonyl and C1-C6 alkylsulfonylamino;
R′ is selected from hydrogen; unsubstituted or hydroxy substituted C1-C6 alkyl; unsubstituted phenyl or phenyl substituted with fluorine, chlorine or bromine and/or with one or more methoxy groups; phenyl(C1-C6)alkyl, in which the phenyl is unsubstituted or substituted with fluorine, chlorine or bromine and/or with one or more methoxy groups, and the C1-C6 alkyl is unsubstituted or hydroxy substituted; —C(O)—(C1-C6)alkyl in which the alkyl is unsubstituted or hydroxy substituted; —C(O)-phenyl in which the phenyl is unsubstituted or substituted with fluorine, chlorine or bromine and/or substituted with one or more methoxy groups; —C(O)—(C1-C6)alkyl-phenyl in which the phenyl is unsubstituted or substituted with fluorine, chlorine or bromine and/or substituted with one or more methoxy groups and the C1-C6 alkyl is unsubstituted or hydroxy substituted; and phenylsulfonyl in which the phenyl is unsubstituted or substituted with fluorine, chlorine or bromine and/or substituted with one or more methoxy groups;
if Q1 is N—R′, R is absent;
if Q1 represents is N, CH, C—R1 or C—X, R is selected from hydrogen; unsubstituted or hydroxy substituted C1-C6 alkyl; unsubstituted phenyl or phenyl substituted with fluorine, chlorine or bromine and/or with one or more methoxy groups substituted phenyl; phenyl(C1-C6)alkyl in which the phenyl is unsubstituted or substituted with fluorine, chlorine or bromine and/or with one or more methoxy groups, and the C1-C6 alkyl is unsubstituted or hydroxy substituted; —C(O)—(C1-C6)alkyl in which the alkyl is unsubstituted or hydroxy substituted; —C(O)-phenyl in which the phenyl is unsubstituted or substituted with fluorine, chlorine or bromine and/or with one or more methoxy groups; —C(O)—(C1-C6)alkyl-phenyl in which the phenyl is unsubstituted or substituted with fluorine, chlorine or bromine and/or substituted with one or more methoxy groups and wherein the C1-C6 alkyl is unsubstituted or hydroxy substituted; and phenylsulfonyl in which the phenyl is unsubstituted or substituted with fluorine, chlorine or bromine and/or substituted with one or more methoxy groups;
X is a group of general formula X2 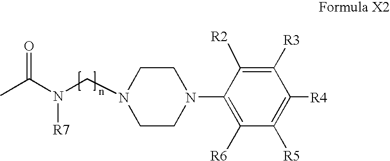
wherein
n has the value of 2, 3, 4 or 5,
R2, R3, R4, R5, R6 and R7 are independently selected from hydrogen; hydroxy; fluorine; chlorine; bromine; trifluormethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methyoxy groups; phenyl(C1-C6)alkyl in which the phenyl is unsubstituted or substituted with fluorine, chlorine or bromine and/or with one or more methoxy groups, and the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl in which the alkyl is unsubstituted or hydroxy substituted; —C(O)-phenyl in which the phenyl is unsubstituted or substituted with fluorine, chlorine or bromine and/or with one or more methoxy groups; —C(O)—(C1-C6)alkyl-phenyl in which the phenyl is
unsubstituted or substituted with fluorine, chlorine or bromine and/or with one or more methoxy groups, and the C1-C6 alkyl is unsubstituted or hydroxy substituted; C1-C6 alkyloxycarbonyl in which the alkyl is unsubstituted or hydroxy substituted; phenyl(C1-C6)alkyloxycarbonyl in which the phenyl is unsubstituted or substituted with fluorine, chlorine or bromine and/or with one or more methoxy groups, and the C1-C6 alkyl is unsubstituted or hydroxy substituted; C1-C6 alkylaminosulfonyl and C1-C6 alkylsulfonylamino, or two vicinal residues R2, R3, R4, R5 and R6 together with the C-atoms of the phenyl ring to which they are bonded form an oxygen-containing 5-, 6- or 7-membered ring, and
R7 is C1-C6 alkyl or hydrogen.

3. The compound according to claim 1, wherein Y is a group —(CH₂)ₙ— and n is 4 or 5.

4. The compound according to claim 1, wherein R7 is H.

5. The compound according to claim 1, comprising a compound having general formula II 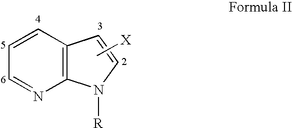
a physiologically acceptable salt thereof, or an enantiomer, diastereomer, or tautomer thereof,
wherein
X is linked to position 2 or 3 of the pyrrolo[2,3-b]pyridine; and
the pyrrolo[2,3-b]pyridine optionally carries R1 at one or more of positions 2, 3, 4, 5 and 6 excluding a position occupied by X.

6. The compound according to claim 5, wherein X is linked to position 2 of the pyrrolo[2,3-b]pyridine.

7. The compound according to claim 5, wherein X is linked to position 3 of the pyrrolo[2,3-b]pyridine.

8. The compound according to claim 5, wherein R is a hydrogen atom, a methyl group or a phenylsulfonyl.

9. The compound according to claim 5, wherein X is a group of general formula X2 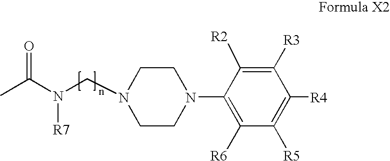
wherein
n is 4 or 5.

10. The compound according to claim 5, wherein at least one of R2 and R3 is selected from a chlorine, fluorine, methoxy, ethoxy, propoxy, methyl, ethyl and propyl.

11. The compound according to claim 5, selected from
N-(4-(4-(2-methoxyphenyl)piperazin-1-yl)butyl)-1H-pyrrolo[2,3-b]pyridin-2-ylcarbamide,
N-(4-(4-(2-methoxyphenyl)piperazin-1-yl)butyl)-1-phenylsulfonyl-1H-pyrrolo[2,3-b]pyridin-2-ylcarbamide,
N-(4-(4-(2,3-dichlorophenyl)piperazin-1-yl)butyl)-1H-pyrrolo[2,3-b]pyridin-2-ylcarbamide,
N-(4-(4-(2,3-dichlorophenyl)piperazin-1-yl)butyl)-1-phenylsulfonyl-1H-pyrrolo[2,3-b]pyridin-2-ylcarbamide,
N-(4-(4-(2,3-dichlorophenyl)piperazin-1-yl)butyl)-1H-pyrrolo[2,3-b]pyridin-3-ylcarbamide,
N-(4-(4-(2-methoxyphenyl)piperazin-1-yl)butyl)-1-methyl-1H-pyrrolo[2,3-b]pyridin-3-ylcarbamide,
N-(4-(4-(2,3-dichlorophenyl)piperazin-1-yl)butyl)-1-phenylsulfonyl-1H-pyrrolo[2,3-b]pyridin-3-ylcarbamide,
N-(4-(4-(2-methoxyphenyl)piperazin-1-yl)butyl)-1-phenylsulfonyl-1H-pyrrolo[2,3-b]pyridin-3-ylcarbamide,
N-(4-(4-(2-ethoxyphenyl)piperazin-1-yl)butyl)-1-phenylsulfonyl-1H-pyrrolo[2,3-b]pyridin-3-ylcarbamide,
N-(4-(4-(2,3-dimethylphenyl)piperazin-1-yl)butyl)-1-phenylsulfonyl-1H-pyrrolo[2,3-b]pyridin-3-ylcarbamide,
N-(4-(4-(dihydrobenzofuran-7-yl)piperazin-1-yl)butyl)-1H-pyrrolo[2,3-b]pyridin-2-ylcarbamide,
N-(4-(4-(dihydrobenzofuran-7-yl)piperazin-1-yl)butyl)-1-phenylsulfonyl-1H-pyrrolo[2,3-b]pyridin-2-ylcarbamide,
N-(4-(4-(chroman-8-yl)piperazin-1-yl)butyl)-1-pyrrolo[2,3-b]pyridin-2-ylcarbamide,
N-(4-(4-(chroman-8-yl)piperazin-1-yl)butyl)-1-phenylsulfonyl-1H-pyrrolo[2,3-b]pyridin-2-ylcarbamide,
N-(4-(4-(2,3,4,5-tetrahydrobenzo[b]oxepin-9-yl)piperazin-1-yl)butyl)-1H-pyrrolo[2,3-b]pyridin-2-ylcarbamide,
N-(4-(4-(2,3,4,5-tetrahydrobenzo[b]oxepin-9-yl)piperazin-1-yl)butyl)-1-phenylsulfonyl-1H-pyrrolo[2,3-b]pyridin-2-ylcarbamide,
N-(4-(4-(dihydrobenzofuran-7-yl)piperazin-1-yl)butyl)-1H-pyrrolo[2,3-b]pyridin-3-ylcarbamide,
N-(4-(4-(dihydrobenzofuran-7-yl)piperazin-1-yl)butyl)-1-phenylsulfonyl-1H-pyrrolo[2,3-b]pyridin-3-ylcarbamide,
N-(4-(4-(chroman-8-yl)piperazin-1-yl)butyl)-1H-pyrrolo[2,3-b]pyridin-3-ylcarbamide,
N-(4-(4-(chroman-8-yl)piperazin-1-yl)butyl)-1-phenylsulfonyl-1H-pyrrolo[2,3-b]pyridin-3-ylcarbamide,
N-(4-(4-(2,3,4,5-tetrahydrobenzo[b]oxepin-9-yl)piperazin-1-yl)butyl)-1H-pyrrolo[2,3-b]pyridin-3-ylcarbamide,
N-(4-(4-(2,3,4,5-tetrahydrobenzo[b]oxepin-9-yl)piperazin-1-yl)butyl)-1-phenylsulfonyl-1H-pyrrolo[2,3-b]pyridin-3-ylcarbamide, and
physiologically acceptable salts thereof.

12. The compound according to claim 1, having general formula IIIa or IIIb 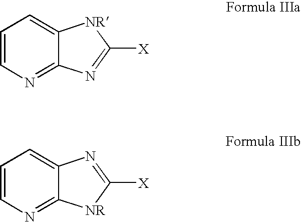
wherein
the imidazo[4,5-b]pyridine optionally carries in the A-ring one or more substituents R1, as;

13. The compound according to claim 12, wherein R is a hydrogen atom or a phenylsulfonyl.

14. The compound according to claim 12, wherein X is a group of general formula X2 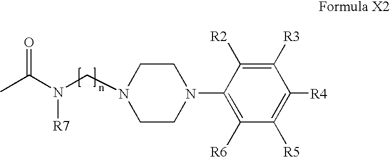
wherein
n is 4 or 5;
R2, R3, R4, R5, R6 and R7 are independently selected from hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl in which the phenyl is unsubstituted or substituted with fluorine, chlorine or bromine and/or with one or more methoxy groups, and the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl in which the alkyl is unsubstituted or hydroxy substituted; —C(O)-phenyl, in which the phenyl is unsubstituted or substituted with fluorine, chlorine or bromine and/or with one or more methoxy groups; —C(O)—(C1-C6)alkyl-phenyl in which the phenyl is unsubstituted or substituted with fluorine, chlorine or bromine and/or with one or more methoxy groups, and the C1-C6 alkyl is unsubstituted or hydroxy substituted; C1-C6 alkyloxycarbonyl in which the alkyl is unsubstituted or hydroxy substituted; phenyl(C1-C6)alkyloxycarbonyl in which the phenyl is unsubstituted or substituted with fluorine, chlorine or bromine and/or with one or more methoxy groups, and the C1-C6 alkyl is unsubstituted or hydroxy substituted; C1-C6 alkylaminosulfonyl and C1-C6 alkylsulfonylamino, or two vicinal residues R2, R3, R4, R5 and R6 together with the C-atoms of the phenyl ring to which they are bonded form an oxygen-containing 5-, 6- or 7-membered ring; and
R7 is C1-C6 alkyl or hydrogen.

15. The compound according to claim 12, wherein at least one of R2 and R3 is selected from chlorine, fluorine, methoxy, ethoxy, propoxy, methyl, ethyl and propyl.

16. A compound according to claim 12 selected from
N-(4-(4-(2-methoxyphenyl)piperazin-1-yl)butyl)-3H-imidazo[4,5-b]pyridin-2-ylcarbamide,
N-(4-(4-(2,3-dichlorophenyl)piperazin-1-yl)butyl)-3H-imidazo[4,5-b]pyridin-2-ylcarbamide,
N-(4-(4-(2-chlorophenyl)piperazin-1-yl)butyl)-3H-imidazo[4,5-b]pyridin-2-ylcarbamide,
N-(4-(4-(2,3-dimethylphenyl)piperazin-1-yl)butyl)-3H-imidazo[4,5-b]pyridin-2-ylcarbamide,
N-(4-(4-(4-fluorophenyl)piperazin-1-yl)butyl)-3H-imidazo[4,5-b]pyridin-2-ylcarbamide,
N-(4-(4-(2,3-dihydrobenzofuran-7-yl)piperazin-1-yl)butyl)-3H-imidazo[4,5-b]pyridin-2-ylcarbamide,
N-(4-(4-(chroman-8-yl)piperazin-1-yl)butyl)-3H-imidazo[4,5-b]pyridin-2-ylcarbamide, and
N-(4-(4-(2,3,4,5-tetrahydrobenzo[b]oxepin-9-yl)piperazin-1-yl)butyl)-3H-imidazo[4,5-b]pyridin-2-ylcarbamide, and
physiologically acceptable salts thereof.

17. The compound according to claim 1, having general formula IV 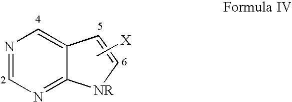
wherein
X is linked to position 5 or 6 of formula IV;
the pyrrolo[2,3-d]pyrimidine optionally carries R1, at one or more of positions 2, 4, 5, and 6 excluding a position occupied by X; and

18. The compound according to claim 17, wherein R is hydrogen, phenylsulfonyl, unsubstituted phenyl, or phenyl substituted with one or more halogen atoms.

19. The compound according to claim 17, wherein the pyrrolo[2,3-d]pyrimidine is unsubstituted or carries one or two substituents R1 selected from hydroxy and C1-C3 alkyl.

20. The compound according to claim 17, wherein X is a group of formula X2, 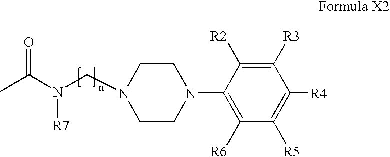
wherein
n is 4 or 5; and
R2, R3, R4, R5, R6 and R7 are independently selected from hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl in which the phenyl is unsubstituted or substituted with fluorine, chlorine or bromine and/or with one or more methoxy groups, and the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl in which the alkyl is unsubstituted or hydroxy substituted; —C(O)-phenyl, in which the phenyl is unsubstituted or substituted with fluorine, chlorine or bromine and/or with one or more methoxy groups; —C(O)—(C1-C6)alkyl-phenyl in which the phenyl is unsubstituted or substituted with fluorine, chlorine or bromine and/or with one or more methoxy groups, and the C1-C6 alkyl is unsubstituted or hydroxy substituted; C1-C6 alkyloxycarbonyl in which the alkyl is unsubstituted or hydroxy substituted; phenyl(C1-C6)alkyloxycarbonyl in which the phenyl is unsubstituted or substituted with fluorine, chlorine or bromine and/or with one or more methoxy groups, and the C1-C6 alkyl is unsubstituted or hydroxy substituted; C1-C6 alkylaminosulfonyl and C1-C6 alkylsulfonylamino, or two vicinal residues R2, R3, R4, R5 and R6 together with the C-atoms of the phenyl ring to which they are bonded form an oxygen-containing 5-, 6- or 7-membered ring; and
R7 is C1-C6 alkyl or hydrogen.

21. The compound according to claim 17, wherein at least one of R2 and R3 is selected from chlorine, fluorine, methoxy, ethoxy, propoxy, methyl, ethyl and propyl.

22. The compound according to claim 17, selected from
N-(4-(4-(2,3-dichlorophenyl)piperazin-1-yl)butyl)-5-methyl-4-hydroxy-7-phenyl-7H-pyrrolo[2,3-d]pyrimidin-6-ylcarbamide and tautomers thereof,
N-(4-(4-(2-methoxyphenyl)piperazin-1-yl)butyl)-5-methyl-4-hydroxy-7-phenyl-7H-pyrrolo[2,3-d]pyrimidin-6-ylcarbamide and tautomers thereof,
N-(4-(4-(2,3-dihydrobenzofuran-7-yl)piperazin-1-yl)butyl)-5-methyl-4-hydroxy-7-phenyl-7H-pyrrolo[2,3-d]pyrimidin-6-ylcarbamide and tautomers thereof,
N-(4-(4-(chroman-8-yl)piperazin-1-yl)butyl)-5-methyl-4-hydroxy-7-phenyl-7H-pyrrolo[2,3-d]pyrimidin-6-ylcarbamide and tautomers thereof,
N-(4-(4-(2,3,4,5-tetrahydrobenzo[b]oxepin-9-yl)piperazin-1-yl)butyl)-5-methyl-4-hydroxy-7-phenyl-7H-pyrrolo[2,3-d]pyrimidin-6-ylcarbamide and tautomers thereof, and
physiologically acceptable salts thereof.

23. A pharmaceutical composition comprising a compound according to claim 1 and a pharmaceutically acceptable adjuvant.

24. A pharmaceutical composition comprising a compound according to claim 5 and a pharmaceutically acceptable adjuvant.

25. A pharmaceutical composition comprising a compound according to claim 12 and a pharmaceutically acceptable adjuvant.

26. A pharmaceutical composition comprising a compound according to claim 17 and a pharmaceutically acceptable adjuvant.

27. A method for treating a disease state in a subject comprising administering to the subject a compound according to claim 1, wherein the disease state is marked by impaired dopamine metabolism and/or abnormal serotonin-5-HT1a signal transmission.

28. A method for treating a disease state in a subject comprising administering to the subject a compound according to claim 1, wherein the disease state is one or more of schizophrenia, depressive disorders, L-dopa- or neuroleptic drug-induced motor disturbances, Parkinson's disease, Segawa syndrome, restless leg syndrome, hyperprolactinemia, hyperprolactinomia, attention deficit hyperactivity syndrome (ADHS) and urinary incontinence.

29. A method for treating or preventing a central nervous system disease or a urinary tract disorder in a subject comprising administering to the subject a compound according to claim 1.

30. A method for treating a disease state in a subject comprising administering to the subject a compound according to claim 5, wherein the disease state is marked by impaired dopamine metabolism and/or abnormal serotonin-5-HT1a signal transmission.

31. A process for preparing a compound according to claim 1, comprising the steps of
reacting an acid derivative A 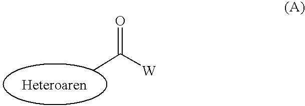
with a free base of general formula C 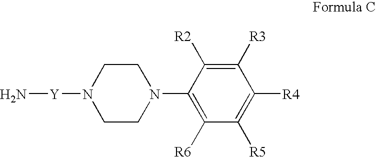
wherein:
W is selected from OH, Cl, Br and a group 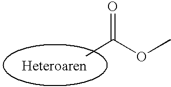
Heteroarene is a group selected from 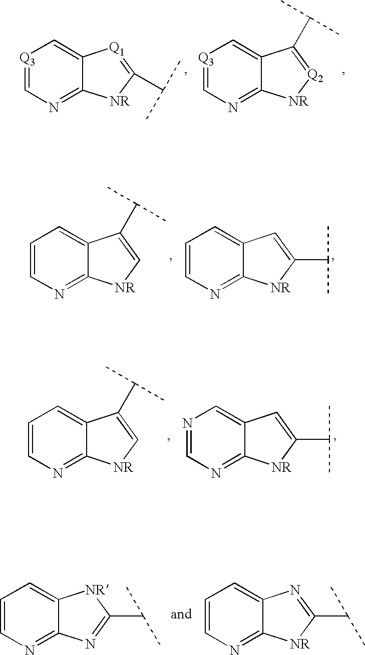
wherein
the crossed-through bond for the heteroarenes is a bonding of the —C(O)—W group to a ring-forming C-atom of the 5-membered ring of the heteroarene;
the heteroarenes are optionally substituted with one or more R1; and
if W is a hydroxy group, the appropriate acid group is activated by addition of one or more activation reagents prior to reaction with the free base of general formula C.
